SEQUENCE LISTING (18 sequences)

SEQ ID NO: 1 - DNA, 20 bp, Artificial Sequence
  Primer ABCG2F
ccatagccacaggccaaagt

SEQ ID NO: 2 - DNA, 20 bp, Artificial Sequence
  Primer ABCG2R
gggccacatgattcttccac

SEQ ID NO: 3 - DNA, 21 bp, Artificial Sequence
  Primer ABCB1bF
tgatcatcagcaacagcagtc

SEQ ID NO: 4 - DNA, 21 bp, Artificial Sequence
  Primer ABCB1bR
tgaaacctggatgtaggcaac

SEQ ID NO: 5 - DNA, 19 bp, Artificial Sequence
  Primer ABCB2F
ctcttgccttggggaaatg

SEQ ID NO: 6 - DNA, 20 bp, Artificial Sequence
  ABCB2R
ctgtgctggctatggtgaga

SEQ ID NO: 7 - DNA, 20 bp, Artificial Sequence
  Primer ABCC7F
gacactttgcttgccctgag

SEQ ID NO: 8 - DNA, 20 bp, Artificial Sequence
  Primer ABCC7R
aagaatcccacctgctttca

SEQ ID NO: 9 - DNA, 21 bp, Artificial Sequence
  Primer ABCA5F
ttctatgtcctcctggctgtg

SEQ ID NO: 10 - DNA, 20 bp, Artificial Sequence
  Primer ABCA5R
tgaccaatacgatggcttca

SEQ ID NO: 11 - DNA, 21 bp, Artificial Sequence
  Primer ABCA3F
ttatgccctcctactggtgtg

SEQ ID NO: 12 - DNA, 21 bp, Artificial Sequence
  Primer ABCA3R
cttgtccttattgcccacttg

SEQ ID NO: 13 - DNA, 21 bp, Artificial Sequence
  Primer ABCB1aF
ccagcagtcagtgtgcttaca

SEQ ID NO: 14 - DNA, 21 bp, Artificial Sequence
  Primer ABCB1aR
gccactccatggataatagca

SEQ ID NO: 15 - DNA, 21 bp, Artificial Sequence
  Primer Beta-actinF
tcctgtggcatccatgaaact

SEQ ID NO: 16 - DNA, 21 bp, Artificial Sequence
  Primer Beta-actinR
gaagcacttgcggtgcacgat

SEQ ID NO: 17 - DNA, 20 bp, Artificial Sequence
  Primer Cre mice Cre F
cggtcgatgcaacgagtgat

SEQ ID NO: 18 - DNA, 20 bp, Artificial Sequence
  Primer Cre R
ccaccgtcagtacgtgagat

(19) United States
(12) Patent Application Publication
(10) Pub. No.: US 2020/0017835 A1
(43) Pub. Date: Jan. 16, 2020

(54) IDENTIFICATION, ISOLATION, AND THERAPEUTIC USES OF ENDOTHELIAL STEM CELLS THAT EXPRESS THE ABCG2+ SURFACE MARKER

(71) Applicant: Indiana University Research and Technology Corporation, Indianapolis, IN (US)

(72) Inventors: Mervin C. Yoder, Indianapolis, IN (US); Yang Lin, New York, NY (US)

(73) Assignee: Indiana University Research and Technology Corporation, Indianapolis, IN (US)

(21) Appl. No.: 16/509,902

(22) Filed: Jul. 12, 2019

Related U.S. Application Data
(60) Provisional application No. 62/697,895, filed on Jul. 13, 2018.

(51) Int. Cl. C12N 5/071; C07K 16/28; A61K 35/44; A61P 9/10

(52) U.S. Cl. CPC C12N 5/0692; C07K 16/28; C12N 2509/10; A61P 9/10; A61K 35/44

(57) ABSTRACT

Methods are provided for the isolation, expansion, enrichment, and transplantation of endothelial stem cells from blood vessels and induced pluripotent stem cells via the use of ABCG2 cell surface marker. The ability of the endothelial stem cells to expand in vitro and be subsequently implanted in vivo to generate new blood vessels provides a therapeutic hope for patients with numerous cardiovascular disorders (peripheral arterial disease, critical limb ischemia, ischemic retinopathies, acute ischemic injury to kidney, and myocardial infarction) where the lack of sufficient blood vessel forming ability in the patient limits their regenerative capacity.

### CROSS-REFERENCE TO RELATED APPLICATIONS

The present application claims priority to U.S. Provisional Patent Application No. 62/697,895, filed Jul. 13, 2018, entitled IDENTIFICATION, ISOLATION, AND THERAPEUTIC USES OF ENDOTHELIAL STEM CELLS THAT EXPRESS THE ABCG2+ SURFACE MARKER, the complete disclosure of which is incorporated by reference herein in its entirety.

### REFERENCE TO SEQUENCE LISTING

This application contains a Sequence Listing submitted via EFS-web which is hereby incorporated by reference in its entirety for all purposes. The ASCII copy, created on Jul. 11, 2019, is named IURTC_2019_005_01_US_ST25.txt and is 3.61 KB in size.

### FIELD

Aspects of the present disclosure include materials and methods for identifying, at least partially isolating, proliferation in vitro and/or in vivo endothelial stem cells which express the ABCG+2 surface protein and using these cells to generate vascular tissue and/or to treat human and animal diseases and/or defects.

### BACKGROUND

All mammals possess a blood vascular system lined with endothelial cells (EC) that provide a dynamic interface between blood and surrounding tissues, regulate nutrient, waste, and blood cell traffic, and participate in regulating hemostasis, inflammation, and angiogenesis. While thousands of articles have been published on angiogenic growth mechanisms, to date, the specific cellular mechanisms for the replacement of damaged, diseased, or senescent vascular EC in intact blood vessels is unclear. It is well known that cells from many tissue lineages, like hematopoietic cells and intestine epithelial cells, are maintained by lineage-specific stem cells that can self-renew and differentiate into mature progeny¹⁻³, but the evidence for endothelial stem cells is relatively nascent. Some EC that can give rise to robust in vitro EC colonies and display vasculogenic properties have been identified from mammalian blood vessels⁴⁻⁷ or from circulating blood⁸.

We and others have used the ability of cells to efflux the DNA dye Hoechst 33342 (such cells are called the side population, SP) to isolate EC with clonogenic and vasculogenic stem cell-like properties^{4,9}, but this phenotype is based on function rather than a cell surface marker and is therefore not feasible to use to prospectively identity these cells in vivo. Recently, several groups have reported the identification of immature EC possessing proliferative potential in selected developmental stages of murine blood vessel development via the differential expression of specific cell surface markers^{7,10-15}. However, whether these EC fulfill all the criteria of unipotent vascular endothelial stem cells (VESC) including clonal proliferative potential, ability to self-renew, contribution to multiple blood vessel compartments (artery, vein, capillary) upon transplantation, and long-term contributions to vessel compartments via fate mapping analysis, has not been thoroughly tested. In addition, putative murine VESC markers have not been validated to isolate VESC in the human system.

Most organs and tissues are maintained lifelong by resident stem cells, however, it is unclear if stem cells replenish vascular endothelial cells. Here, we report that the ATP cassette transporter Abcg2 labels murine resident vascular endothelial stem cells that display clonal proliferative potential and blood vessel forming ability to give rise to artery, vein, and capillary EC, in addition to displaying self-renewal activity in vivo. Transcriptome analysis reveals that Abcg2-expressing endothelial stem cells from different tissues express a common gene expression signature involved in angiogenesis and proliferation regulation in addition to distinct tissue-specific expression patterns. ABCG2 also serves as a marker that labels human resident vascular endothelial stem cells. These results are the first to establish that a single prospective marker identifies vascular endothelial stem cells in mouse and man and hold promise to provide new cell therapies for repair of damaged vessels in patients with endothelial dysfunction.

### SUMMARY

According to one embodiment, the present disclosure provides a method for identifying and enriching a population of endothelial stem cells, including the steps of: contacting a population of cells that includes endothelial stem cells with an agent, wherein the agent selectively binds to the cell surface marker ABCG2+; and recovering at least a portion of endothelial stem cells that bind to the agent which selectively bind to ABCG2+. In some aspects of this embodiment, the agent is an antibody. In some aspects of this embodiment, the antibody is linked to a bead. In some aspects of this embodiment, the bead is magnetic. In some aspects, the present disclosure provides a method that further includes isolating at least one endothelial stem cell that exhibits the ABCG2+ surface marker. In other aspects, the present disclosure provides a method that includes the step of creating a population of cells enriched in the endothelial stem cells that exhibits the ABCG2+ surface marker. In other aspects, the present disclosure further includes the step of culturing the endothelial stem cells that express the ABCG2+ surface marker, in vitro. In other aspects, the present disclosure provides a method wherein the endothelial stem cell(s) that exhibits the ABCG2+ surface marker blood vessel cells are derived from human umbilical artery, umbilical vein, or saphenous vein. In other aspects, the present disclosure provides a method wherein the blood vessel cells are derived from murine umbilical artery, umbilical vein, or saphenous vein.

According to one embodiment, the present disclosure provides a method of identify endothelial stem cell that exhibits the ABCG2+ surface marker.

According to one embodiment, the present disclosure provides a method for the ex vivo expansion of endothelial stem cells, including the steps of: providing at least one endothelial stem cell that exhibits the ABCG2+ surface marker; and culturing the at least one endothelial stem cell that exhibits the ABCG2+ surface marker under condition that increase the number of the endothelial stem cells that exhibits the ABCG2+ surface marker cells. In some embodiments, the culturing step is carried out in the presence of OP9 stromal cells.

According to one embodiment, the present disclosure provides a method of transplanting ex-vivo expanded endothelial stem cells according to any of the preceding or succeeding paragraphs into a recipient that would benefit from blood vessel forming, the method including: obtaining a population of ABCG2+ endothelial stem cells; and transplanting the population into a living human or animal. In some embodiments, the method further includes transplanting a portion of stromal cells with the population of ABCG2+ endothelial stem cells. In some embodiments, the method further includes the steps of: removing a portion of the transplanted population from a recipient; culturing the population of cells; and transplanting the population into the same or a different recipient.

According to one embodiment, the present disclosure provides a medicament for the treatment of a patent, the medicament including at least one ABCG2+ endothelial stem cells or a population ABCG2+ endothelial stem cells. In some embodiments the ABCG2+ endothelial stem cells are collected and used to create a population of cells enriched in ABCG2+ endothelial stem cells. In some embodiments, the population ABCG2+ endothelial stem cells is expanded ex vivo. In some embodiments, the medicament further includes at least one regent that promotes the stabilized and or promotes the growth of the ABCG2+ endothelial stem cells. In some embodiments, the medicament further includes a gel, in some instances, the gel is a collagen gel.

According to one embodiment, the present disclosure provides a method of treating a patient, the method including the steps of: administering at least one dose of a therapeutically effective amount of ABCG2+ endothelial stem cells to a human or animal patient. In some embodiments, the ABCG2+ endothelial stem cells are suspended in collagen gel. In some embodiment the cells are suspended in a matrix that does not include collagen or in a container suitable for the delivery of the cells into the body of a patient. In some embodiments, the therapeutically effective amount of ABCG2+ endothelial stem cells is on the order of more than two million cells per milliliter of collagen gel.

In some embodiments, the human or animal patient has been diagnosed with a condition that can benefit from development of an increase in vascular tissue. In some embodiments, the human or animal patient exhibits at least one disease or defect selected from the groups consisting of peripheral arterial disease, critical limb ischemia, ischemic retinopathies, acute ischemic injury to kidney, and myocardial infarction.

While multiple embodiments are disclosed, still other embodiments of the present disclosure will become apparent to those skilled in the art from the following detailed description, which shows and describes illustrative embodiments of the present disclosure. Accordingly, the drawings and detailed description are to be regarded as illustrative in nature and not restrictive.

### BRIEF DESCRIPTION OF THE SEQUENCES

SEQ. ID NO. 1 5′-CCATAGCCACAGGCCAAAGT-3′ ABCG2F

SEQ. ID NO. 2 5′-GGGCCACATGATTCTTCCAC-3′ ABCG2R

SEQ. ID NO. 3 5′-TGATCATCAGCAACAGCAGTC-3′ ABCB1bF

SEQ. ID NO. 4 5′-TGAAACCTGGATGTAGGCAAC-3′ ABCB1bR

SEQ. ID NO. 5 5′-CTCTTGCCTTGGGGAAATG-3′ ABCB2F

SEQ. ID NO. 6 5′-CTGTGCTGGCTATGGTGAGA-3′ ABCB2R

SEQ. ID NO. 7 5′-GACACTTTGCTTGCCCTGAG-3′ ABCC7F

SEQ. ID NO. 8 5′-AAGAATCCCACCTGCTTTCA-3′ ABCC7R

SEQ. ID NO. 9 5′-TTCTATGTCCTCCTGGCTGTG-3′ ABCA5F

SEQ. ID NO. 10 5′-TGACCAATACGATGGCTTCA-3′ ABCA5R

SEQ. ID NO. 11 5′-TTATGCCCTCCTACTGGTGTG-3′ ABCA3F

SEQ. ID NO. 12 5′-CTTGTCCTTATTGCCCACTTG-3′ ABCA3R:

SEQ. ID NO. 13 5′-CCAGCAGTCAGTGTGCTTACA-3′ ABCB1aF

SEQ. ID NO. 14 5′-GCCACTCCATGGATAATAGCA-3′ ABCB1aR

SEQ. ID NO. 15 5′-TCCTGTGGCATCCATGAAACT-3′ Beta-actinF

SEQ. ID NO. 16 5′-GAAGCACTTGCGGTGCACGAT-3′ Beta-actinR

SEQ. ID NO. 17 5′-CGG TCG ATG CAA CGA GTG AT-3′ Cre mice: Cre F

SEQ. ID NO. 18 5′-CCA CCG TCA GTA CGT GAG AT-3′ Cre R

### BRIEF DESCRIPTION OF THE FIGURES

FIG. 1A. Abcg2-expressing VESC contribute to vessel growth in vivo during development. a, Schematics of lineage tracing experiment using ABCG2TT mice to test the contribution of P1 Abcg2-expressing VESC to vasculature development in multiple organs.

FIG. 1B. P1 (top panels, representative of 5 animals) and 3-week-old (bottom panels, representative of 7 animals) ABCG2TT mice heart after tamoxifen injection at P0. Green, CD31; Red, TdTomato; Blue, ERG. Single TdTomato+ EC (arrows) at P1 developed into continues TdTomato+ vessels (star) at P21.

FIG. 1C. Representative flow cytometry chart (from 5 P1 and 7 P21 mice) showing the increase of the percentage of TdTomato+ EC from P1 (top) to P21 (bottom) heart in ABCG2TT mice (left panels) after P0 tamoxifen injection.

FIG. 1D. Quantitation of TdTomato+ (red bars) and TdTomato− (blue bars) EC number in P1 (left) and P21 (right) ABCG2TT mouse heart after tamoxifen injection at P0. Data represent mean±s.d. p values, two-tailed unpaired t-test. (P1: n=5; P21: n=7. From 2 independent experiments).

FIG. 1E. Quantitation of percentage of TdTomato+ EC in multiple developmental stages of ABCG2TT mice heart after tamoxifen injection at P0. 17 Data represent mean±s.d. (P1: n=5; P3: n=6; P10: n=4; P21: n=7; P56: n=5; P300: n=7; P540: n=3. From 2 independent experiments).

FIG. 1F. Representative pictures (from 7 mice) show the contribution of P0 labeled TdTomato+ Abcg2-expressing VESC to arterial (arrows), venous (dashed arrow) and capillary (arrowhead) of 3-week-old ABCG2TT mouse heart. Green, CD31; Red, TdTomato; Gray, smooth muscle actin α (SMA).

FIG. 1G. Representative pictures showing the contribution of P0 labeled TdTomato+ Abcg2-expressing single EC at P1 skin contribute to large vessel (arrows) and capillary (arrowhead) EC of P7 ABCG2TT mice skin. Green, CD31; Red, TdTomato; Gray, smooth muscle actin α (SMA). Data represents the results derived from 7 mice.

FIG. 2A. Abcg2-expressing VESC have in vitro EC colony forming potential and in vivo vessel forming potential. a, Schematics of in vitro colony forming assay and in vivo vessel formation assay using Abcg2-expressing VESC from ABCG2TT mice.

FIG. 2B. OP9 co-cultured in vitro EC colonies (7 days) derived from TdTomato+ (top panels) or TdTomato− (bottom panels) heart EC of P1 ABCG2TT mice after tamoxifen injection at P0. Data represents results derived from 4 mice.

FIG. 2C. and FIG. 2D. Quantitation of frequencies of colony forming cells in (FIG. 2C) lung (c) and (FIG. 2D) heart (d) TdTomato+ (red bars) and TdTomato− (blue bars) EC from P1 ABCG2TT mice after tamoxifen injection at P0. Data represent mean±s.d. p values, two-tailed unpaired t-test. (n=4 mice from 2 independent experiments).

FIG. 2E. Representative pictures of P1 ABCG2TT heart EC (P0 tamoxifen injected) derived vessels 2 weeks after collagen gel plug transplantation (TdTomato, red, CD31,). This picture is a representative of 3 experiments using 3 individual mice. Arrow indicates TdTomato+ EC in a large vessel. Dashed arrow indicates TdTomato+ capillary EC. Red, TdTomato. Green, CD31.

FIG. 2F. Comparison of vessel forming potential of TdTomato+ (red bars) and TdTomato− (blue bars) EC from P1 ABCG2TT mice after tamoxifen injection at P0. Data represent mean±s.d. p values, two-tailed unpaired t-test (n=3 mice).

FIG. 2G. Representative picture of P1 ABCG2TT mice heart TdTomato+ VESC secondary colony EC formed blood vessels after secondary transplantation (represents gels derived from 6 individual mice). Red, TdTomato. Green, CD31.

FIG. 3A. Abcg2-expressing VESC maintain adult blood vessels. a, Schematics of lineage tracing experiment using ABCG2TT mice to test the contribution of adult Abcg2− expressing VESC to vasculature development in multiple organs.

FIG. 3B. Representative pictures (from 5 mice) showing the distribution of TdTomato+ VESC (arrows) in 6-week-old heart of ABCG2TT mice 24 hours after tamoxifen injection. Red, TdTomato; Green, CD31; Gray, smooth muscle actin α (SMA).

FIG. 3C. Flow cytometry data showing the percentage of TdTomato+ cells in 6-week-old heart EC of ABCG2TT mice that had received tamoxifen injection 24 hours before the experiment.

FIG. 3D. Frequency of colony forming cells in TdTomato+ and TdTomato− EC from 6-week-old ABCG2TT heart with tamoxifen injected 24 hours before sorting. Data represent mean±s.d. p values, two-tailed unpaired t-test. (n=4 mice).

FIG. 3E. Representative pictures showing 1 day (top panels, from 4 mice) after tamoxifen was injected into 8-week-old adult mice, TdTomato EC were mostly single cells (arrows) while after 6 weeks (bottom panels, from 4 mice) TdTomato+ EC formed clusters that contain several TdTomato+ EC (dashed arrows). Red, TdTomato; Green, CD31; Blue, DAPI.

FIG. 3F. Representative pictures generated from Imaris software showing the clonal expansion of TdTomato+ EC from day1 (after tamoxifen injection) adult mice skin (top panel) to TdTomato+ EC clusters in skin of ABCG2TT mice 6 weeks after tamoxifen injection (bottom panel). Each red object indicates a single TdTomato+ EC or a continuous TdTomato+EC cluster in the vessel. Each sphere indicates a DAPI+ nucleus.

FIG. 3G. Quantitation of the number of nuclei in each continuous TdTomato+ EC cluster from skin of ABCG2TT mice 1 day or 6 weeks after tamoxifen injection at 8-week-old. Data represent mean±s.d. p values, two-tailed unpaired t-test. (1 day: n=143 clusters from 4 mice; 6 weeks: n=177 clusters from 4 mice).

FIG. 3H. Quantitation of percentage of TdTomato+ EC in adult ABCG2TT mice skin 1 day and 12 weeks after tamoxifen injection at 8-week-old. Data represent mean±s. d. (1 day, n=5; 12 weeks, n=5).

FIG. 3I. Quantitation of the recovery of blood flow in ischemia legs of nude mice received TdTomato+ VESC (red) or PBS (blue). Blood flow in uninjured legs were used as references. Data represent mean±s.d. p values, two-tailed unpaired t-test. (n=10 mice from 3 independent experiments).

FIG. 3J. Contribution of TdTomato+ VESC to recipient muscle blood vessels after ischemic injury. Red, TdTomato; Green, isolectinB4 (IB4); Blue, CD31. Data represent results derived from 10 mice.

FIG. 4A. Transcriptome analysis of Abcg2-expressing VESC. a. GO (left panel) and KEGG (right panel) pathway analysis for P1 heart TdTomato+ Abcg2-expressing VESC versus TdTomato− EC from ABCG2TT pups received tamoxifen injection at P0 (n=4 mice).

FIG. 4B. Heatmap showing genes that were commonly upregulated in TdTomato+ EC from all 5 comparisons (TdTomato+ versus TdTomato− EC from P1 heart, P1 lung EC, P6 muscle, 6 week heart, 6 week muscle. All animals received tamoxifen injection 24 hours before collection) and were significant (FDR<0.05) in at least two groups (neonatal heart, adult muscle, n=4; neonatal lung, muscle, adult heart, n=5. adt, adult; neo, neonatal; hrt, heart; lng, lung; mus, muscle; Td, Tdtomato).

FIG. 4C. Comparison of the expression of lung, heart and muscle tissue-specific EC transcription factors (TF) among neonatal heart (hrt), muscle (mus) and lung (lng) TdTomato+ Abcg2-expressing VESC.

FIG. 4D. Jaccard distance analysis of all neonatal heart (hrt), muscle (mus) and lung (lng) TdTomato+ Abcg2-expressing VESC samples based on the expression of lung, heart and muscle tissue-specific EC transcription factors. Smaller number indicates two samples were more similar to each other.

FIG. 5A. Human VESC are labeled by ABCG2. a. Representative pictures (from 3 patients) show the distribution of ABCG2+EC (arrows) in human umbilical artery (top panels) and vein (bottom panels). Right panels show merged picture with ABCG2, CD31 and DAPI. Red, ABCG2; Green, CD31; Blue, DAPI.

FIG. 5B. Flow cytometry data (represents 6 patients) showing the percentage of ABCG2+EC in primary human umbilical artery (HUAEC) or vein (HUVEC) EC.

FIG. 5C. Quantitation of data from FIG. 5B. Data represent mean±s. p values (n=6 patients).

FIG. 5D. ABCG2+EC (arrows) in adult human saphenous vein. Green, human CD31; Red, ABCG2.

FIG. 5E. Percentage of EC colony forming cells in freshly isolated ABCG2+ and ABCG2-CD31+CD45-HUVEC. Data represent mean±s.d. p values, two-tailed paired t-test. (n=7 patients from 5 independent experiments).

FIG. 5F. Single ABCG2+ HUVEC derived blood vessels (represent 4 patients) 2 weeks after transplantation. Cyan, human CD31.

FIG. 5G. Single ABCG2+ HUVEC derived arteries and capillaries 2 weeks after co-transplantation with OP9-DL1. Right panel shows merged picture with CD31 and SMA. Cyan, human CD31; Red, smooth muscle actin α(SMA).

FIG. 6A. (Extended Data FIG. 1). Abcg2 is crucial for the maintenance of EC colony forming cells. a. Representative flow dot plot (from 8 mice) showing side (SP) and main population (MP) cells from 8-week-old adult lung EC (left panel). Adding of verapamil, a calcium channel inhibitor, blocks SP phenotype (right panel).

FIG. 6B. (Extended Data FIG. 1). Representative picture of an alkaline phosphatase stained CD31+EC colony derived from lung CD45-CD31+ side population EC in OP9 co-culture.

FIG. 6C. (Extended Data FIG. 1). qPCR analysis of the expression of seven ATP binding cassette family transporters in lung CD45-CD31+EC SP (red bars) and MP (blue bars). Data represent mean±s.d. p values, two-tailed unpaired t-test. (n=3 mice from 3 independent experiments).

FIG. 6D. (Extended Data FIG. 1). Quantitation of colony forming potential of 8-week-old lung EC side population (SP no Vera, red bar), side population with verapamil (SP with Vera, red stripes bar), main population (MP no Vera, blue bar) and main population with verapamil (MP with Vera, blue stripes bar). Data represent mean±s.d. p values, two-tailed unpaired t-test. (n=4 mice).

FIG. 6E. (Extended Data FIG. 1). Western blot of Abcg2 expression in the kidneys of wild type (WT) and Abcg2 knockout (KO) mice. Beta actin was used as internal control.

FIG. 6F. (Extended Data FIG. 1). Representative pictures (from 6 WT and 6 KO mice) of CD31+EC colonies (indicated by red circles) derived from 10,000 lung CD45-CD31+EC (OP9 co-culture, plated in 1 well of 6 well plate) from P1 wild type FVB (WT, left panel) and Abcg2 knockout (KO, right panel) mice.

FIG. 6G. (Extended Data FIG. 1). Quantitation of numbers of EC colony forming EC in 10,000 (10K) lung CD45-CD31+EC from P1 wild type FVB (WT, red bar), Abcg2 knockout (KO, blue bar) and heterozygous (Het, red stripes bar) mice. Data represent mean±s.d. p values, two-tailed unpaired t-test. (n=6 mice).

FIG. 7A. (Extended Data FIG. 2). Abcg2-expressing VESC contribute to vessel growth in vivo during development. Schematics of lineage tracing experiments using ABCG2TT mice.

FIG. 7B. (Extended Data FIG. 2). qPCR of the expression of Abcg2, Abcb1a, Abcb1b in P1 ABCG2TT mice heart TdTomato+ (red bars) and TdTomato− (blue bars) EC (tamoxifen injection at P0). Data represent mean±s.d. p values, two-tailed unpaired t-test. (n=3 mice).

FIG. 7C. (Extended Data FIG. 2). Representative pictures (from 5 mice) showing the distribution of TdTomato+ EC in the capillaries (top panels, arrowheads), artery (middle panels, arrows) and vein (bottom panels, dashed arrow) of a P1 ABCG2TT mouse heart after tamoxifen injection at P0 (Red, TdTomato, Green, CD31, Gray, smooth muscle actin α+[SMA]).

FIG. 7D. (Extended Data FIG. 2). Representative pictures (from 4 mice) showing TdTomato+ EC in the arteries (arrows), veins (dashed arrows) and capillaries (arrowheads) in P4 ABCG2TT mice retina after tamoxifen injection at P3.

FIG. 7E. (Extended Data FIG. 2). Quantitation of the percentage of TdTomato+ EC in the artery (red bar), vein (blue bar) and capillaries (white bar) in P4 ABCG2TT mice retina after tamoxifen injection at P3. Data represent mean±s.d. (n=3 mice. For each mouse, ERG+ nuclear staining of >200 arterial EC, >200 venous EC and >500 capillary EC from confocal images were analyzed).

FIG. 7F. (Extended Data FIG. 2). Representative pictures (from 4 mice) of TdTomato+ EC in P4 ABCG2TT retina tip (arrows, left and middle panels), stalk (dashed arrow, top right) and phalanx (arrowhead, bottom right) cells after tamoxifen injection at P3. f and g, Red, TdTomato, Green, ERG.

FIG. 7G. (Extended Data FIG. 2). Contribution of P0 labeled TdTomato+ Abcg2− expressing VESC to EC of 300 days old mice heart. Green, CD31; Red, TdTomato. Data represent results derived from 7 mice.

FIG. 7H. (Extended Data FIG. 2). Representative pictures (from 5 mice) show the contribution of P0 labeled TdTomato+ Abcg2-expressing VESC to endothelial cells (EC, arrow) but not endocardium (dashed arrow) of 3-week-old ABCG2TT mice heart. Green, CD31; Red, TdTomato.

FIG. 7I. (Extended Data FIG. 2). Contribution of P0 labeled TdTomato+ Abcg2-expressing VESC to 10 days old ABCG2TT mice retinal vasculatures include arteries (arrow), veins (dashed arrow) and capillaries (arrowhead). Red, TdTomato; Green, isolectin B4 (IB4); Gray, CD31 (represents results derived from 4 individual mice).

FIG. 7J., FIG. 7K., FIG. 7L., FIG. 7M., and FIG. 7N. (Extended Data FIG. 2). contribution of P1 neonatal Abcg2-expressing VESC to 3-week-old ABCG2TT mice vascular EC.

FIG. 7J. (Extended Data FIG. 2). P1 (top panels, represent data from 5 mice) and 3 week old (bottom panels, from 7 mice) ABCG2TT mice lung (j) Tomoxifen was injected at P0. Green, CD31; Red, TdTomato; Blue, ERG.

FIG. 7K. (Extended Data FIG. 2). Quantitation of percentage of TdTomato+ EC in multiple developmental stages of ABCG2TT mice lung (k), after tamoxifen injection at P0. Data represent mean±s.d. (P1: n=5; P3: n=6; P10: n=4; P21: n=7; P56: n=5; P300: n=7; P540: n=3. From 2 independent experiments).

FIG. 7L (Extended Data FIG. 2). P1 (top panels, represent data from 5 mice) and 3 week old (bottom panels, from 7 mice) ABCG2TT mice bone marrow (I). Tomoxifen was injected at P0. Green, CD31; Red, TdTomato; Blue, ERG.

FIG. 7M. (Extended Data FIG. 2). Quantitation of percentage of TdTomato+ EC in multiple developmental stages of ABCG2TT bone marrow (m), after tamoxifen injection at P0. Data represent mean±s.d. (P1: n=5; P3: n=6; P10: n=4; P21: n=7; P56: n=5; P300: n=7; P540: n=3. From 2 independent experiments).

FIG. 7N. (Extended Data FIG. 2). Quantitation of percentage of TdTomato+ EC in multiple developmental stages of ABCG2TT muscle (n) after tamoxifen injection at P0. Data represent mean±s.d. (P1: n=5; P3: n=6; P10: n=4; P21: n=7; P56: n=5; P300: n=7; P540: n=3. From 2 independent experiments).

FIG. 7O (Extended Data FIG. 2). Representative picture (from 3 mice) showing the contribution of P0 labeled Abcg2-expressing VESC to 540-day-old ABCG2TT mouse skin arterial (arrow) and capillary (arrowhead) EC. Red, TdTomato; Green, CD31; gray, smooth muscle actin (SMA); Blue, DAPI.

FIG. 7P. (Extended Data FIG. 2). Representative pictures showing WT ROSATdTomato mice that received tamoxifen at P0 does not have TdTomato+ EC in P540 heart, FIG. 7P (left panel), lung (middle panel) and muscle (right panel). Green, CD31; Red, TdTomato; blue, ERG. capillaries (top panels, arrowheads), artery (middle panels, arrows) and vein (bottom panels, dashed arrow) of a P1 ABCG2TT mouse heart after tamoxifen injection at P0 (Red, TdTomato, Green, CD31, Gray, smooth muscle actin α+[SMA]).

FIG. 8A. (Extended Data FIG. 3). Abcg2-expressing VESC have in vitro EC colony forming potential and in vivo vessel forming potential. a, Percentage of TdTomato+ cells in P1 ABCG2TT mice (P0 tamoxifen injected) lung EC, heart EC (determined by flow cytometry) and percentage of TdTomato+ EC colonies in lung EC, heart EC derived colonies. Data represent mean±s.d. (n=4 mice).

FIG. 8B. (Extended Data FIG. 3). Representative picture of a single P1 ABCG2TT mouse heart TdTomato+ EC derived colony.

FIG. 8C. (Extended Data FIG. 3). Schematic of in vivo vessel forming potential assay for a single P1 ABCG2TT mouse heart TdTomato+ EC.

FIG. 8D. (Extended Data FIG. 3). Blood vessels formed by a single P1 ABCG2TT mouse heart TdTomato+ EC (Red, TdTomato, Green, CD31). This is a representative picture from 6 experiments using cells from 6 individual mice.

FIG. 8E. (Extended Data FIG. 3). Representative picture (from 4 mice) showing the perfusion of P1 ABCG2TT mouse heart TdTomato+ EC derived vessel 2 weeks after transplantation. Red, TdTomato, Green, IsolectinB4 (IB4).

FIG. 8F. (Extended Data FIG. 3). Numbers of TdTomato+ (red bar) and TdTomato− (blue bar) EC from P1 ABCG2TT mice (tamoxifen injection at P0) heart EC collagen gel plug transplantation. Data represent mean±s.d. (n=3 mice).

FIG. 8G. (Extended Data FIG. 3). Quantitation of the average volume of TdTomato+ (red bar) and TdTomato− (blue bar) blood vessels in every 100 μm3gel. Data represent mean±s.d. p values, two-tailed unpaired t-test. (n=3 mice. For each data point, four 509×509×58 μm volumes were randomly selected and imaged from the center area of each retrieved gels 2 weeks after transplantation. The surface areas of TdTomato+ and total CD31+ vessels were measured using Imarissoftware).

FIG. 9A. (Extended Data FIG. 4). Abcg2-expressing VESC are maintained in adult blood vessels. a. Representative picture showing TdTomato+ Abcg2-expressing EC in vein (dashed arrow) and capillary (arrowhead), but now artery (arrow) of adult ABCG2TT mice heart 1 day after tamoxifen injection. Green, CD31; Red, TdTomato; Gray, smooth muscle actin α(SMA).

FIG. 9B. (Extended Data FIG. 4). Representative picture of the distribution of TdTomato+ EC in adult lung 1 day after tamoxifen injection. Green, CD31; Red, TdTomato; Blue, DAPI.

FIG. 9C. (Extended Data FIG. 4). Representative picture of the distribution of TdTomato+ EC in vein (dashed arrow) and capillary (arrowhead), but now artery (arrow) of adult ABCG2TT mice muscle 1 day after tamoxifen injection. Green, CD31; Red, TdTomato; Gray, smooth muscle actin α(SMA).

FIG. 9D. (Extended Data FIG. 4). Frequency of TdTomato+ EC in artery, vein and capillary of adult ABCG2TT mice muscle 1 day after tamoxifen injection. Deep imagine confocal pictures were analyzed by image cytometer to generate this data. Data represent mean±s.d. (n=3 mice).

FIG. 9E. (Extended Data FIG. 4). TdTomato+ Abcg2-expressing EC in vein (top panels, arrow) and capillaries (bottom panel, arrow) of retina. Green, CD31; Red, TdTomato; Gray, smooth muscle actin α(SMA).

FIG. 10A. (Extended Data FIG. 5). Abcg2-expressing VESC maintain adult blood vessels. a. Representative picture (from 4 mice) showing the contribution of Abcg2-expressing adult skin EC to TdTomato+ arterial (arrow), arteriole (dashed arrow) and capillary (arrowhead) EC 6 weeks after tamoxifen injection. Green, CD31; Red, TdTomato; Gray, smooth muscle actin α (SMA).

FIG. 10B. (Extended Data FIG. 5). Quantitation of percentage of TdTomato+EC in adult ABCG2TT mice muscle (b) 1 day, 6 weeks, and 12 weeks after tamoxifen injection in 8 week old mice. Data represent mean±s.d. (1 day, n=5; 6 weeks, n=4; 12 weeks, n=5).

FIG. 10C. (Extended Data FIG. 5). Quantitation of percentage of TdTomato+EC in adult ABCG2TT mice bone marrow (c) 1 day, 6 weeks, and 12 weeks after tamoxifen injection in 8 week old mice. Data represent mean±s.d. (1 day, n=5; 6 weeks, n=4; 12 weeks, n=5).

FIG. 10D. (Extended Data FIG. 5). Representative picture (from 4 mice) showing adult retinal TdTomato+ EC in ABCG2TT mice did not proliferate from 1 day to 6 weeks after tamoxifen injection. Green, CD31; Red, TdTomato; Blue, DAPI.

FIG. 10E. (Extended Data FIG. 5). Quantitation of percentage of TdTomato+ EC in adult ABCG2TT mice heart (e). 1 day, 6 weeks, and 12 weeks after tamoxifen injection at 8 week old. Data represent mean±s.d. (1 day, n=5; 6 weeks, n=4; 12 weeks, n=5).

FIG. 10F. (Extended Data FIG. 5). Quantitation of percentage of TdTomato+ EC in adult ABCG2TT mice heart lung (f). 1 day, 6 weeks, and 12 weeks after tamoxifen injection at 8 week old. Data represent mean±s.d. (1 day, n=5; 6 weeks, n=4; 12 weeks, n=5).

FIG. 11A. (Extended Data FIG. 6). Abcg2-expressing VESC can participate in vessel regeneration after ischemic injury. a. Rescue of blood flow in the legs of nude mice after hind limb ischemia. Representative pictures of blood flow of ischemia induced legs (left of each panel) and uninjured control legs (right of each panel) of nude mice at day 0 of experiment (left panel) or 42 days after injected with PBS (middle panel, 10 mice) or TdTomato+EC (right panels, 10 mice).

FIG. 11B. (Extended Data FIG. 6). Contribution of TdTomato+ EC from 6 week old ABCG2TT mice (tamoxifen injected 24 hours before collection) to the arteries (top panels) and capillaries (bottom panels) of nude mice ischemic leg muscle 42 days after cell injection. Red, TdTomato; Green, smooth muscle actin (SMA); Blue, DAPI.

FIG. 12A. and FIG. 12B. (Extended Data FIG. 7). Comparison of Abcg2-expressing VESC to other putative VESC markers. a, b. Representative flow cytometry chart (from 3 mice) showing in the heart of adult ABCG2TT mice 1 day after tamoxifen injection. The majority of EC express ProcR (a), while CD157+EC and Tomato Abcg2-expressing EC fractions had minimal overlap (b, left panel). Majority of TdTomato+ EC (b, middle panel) and CD157+EC (b, right panel) also express ProcR.

FIG. 12A. (Extended Data FIG. 7). The majority of EC express ProcR (a),

FIG. 12B. (Extended Data FIG. 7). While CD157+EC and Tomato Abcg2-expressing EC fractions had minimal overlap (b, left panel).

FIG. 12C. (Extended Data FIG. 7). Venn diagrams of the percentage of ProcR+, CD157+, and Abcg2-expressing TdTomato+ EC in heart (c) of adult ABCG2TT mice 1 day after tamoxifen injection. Numbers in Venn diagrams show the percentage of each fraction of total EC. Hierarchical figures show the percentage of each sub-fraction from its parental fraction.

FIG. 12D. (Extended Data FIG. 7). Venn diagrams of the percentage of ProcR+, CD157+, and Abcg2-expressing TdTomato+ EC in lung (d), of adult ABCG2TT mice 1 day after tamoxifen injection. Numbers in Venn diagrams show the percentage of each fraction of total EC. Hierarchical figures show the percentage of each sub-fraction from its parental fraction.

FIG. 12E. (Extended Data FIG. 7). Venn diagrams of the percentage of ProcR+, CD157+, and Abcg2-expressing TdTomato+ EC in and skeletal muscle (e) of adult ABCG2TT mice 1 day after tamoxifen injection. Numbers in Venn diagrams show the percentage of each fraction of total EC. Hierarchical figures show the percentage of each sub-fraction from its parental fraction.

FIG. 12F. (Extended Data FIG. 7). Representative flow cytometry dot plot (from 3 mice) showing in the heart of adult ABCG2TT mice 1 day after tamoxifen injection, about half of EC express cKit.

FIG. 12G. (Extended Data FIG. 7). Schematics of lineage tracing experiment using ABCG2TT mice to test the contribution of neonatal Abcg2-expressing VESC to adult CD157+ VESC in multiple organs.

FIG. 12H., FIG. 12I., and FIG. 12J. (Extended Data FIG. 7). Representative flow cytometry data (from 3 mice) and quantitation data (FIG. 12 I, and FIG. 12J) showing the contribution of P0 tamoxifen labeled neonatal Abcg2-expressing VESC to adult heart (FIG. 12 H, and FIG. 12 I) and muscle (FIG. 12J) CD157+ VESC. (FIG. 12 I, and FIG. 12 J.). Data represent mean±s.d. (P7: n=4; P21: n=5; P56: n=6; P540: n=3.)

FIG. 13A. (Extended Data FIG. 8). Transcriptome analysis of Abcg2-expressing VESC. Numbers of genes that were significantly (FDR<0.05) up- or down-regulated in TdTomato+ Abcg2-expressing VESC versus TdTomato− EC from ABCG2TT mice EC 24 hours after tamoxifen injection.

FIG. 13B. (Extended Data FIG. 8). GO (left panels) and KEGG (right panels) pathway analysis for TdTomato+ Abcg2-expressing VESC versus TdTomato− EC. b, p6 muscle.

FIG. 13C. (Extended Data FIG. 8). GO (left panels) and KEGG (right panels) pathway analysis for TdTomato+ Abcg2-expressing VESC versus TdTomato− EC c, p1 lung.

FIG. 13D. (Extended Data FIG. 8). GO (left panels) and KEGG (right panels) pathway analysis for TdTomato+ Abcg2-expressing VESC versus TdTomato− EC. d, 6 week heart.

FIG. 13E. (Extended Data FIG. 8). GO (left panels) and KEGG (right panels) pathway analysis for TdTomato+ Abcg2-expressing VESC versus TdTomato− EC. 6 week muscle.

FIG. 13F., and FIG. 13G. (Extended Data FIG. 8). Heatmaps of angiogenesis FIG. 13F. and endothelial to mesenchymal transition FIG. 13G genes. Each square shows the comparison of average gene expression between TdTomato+ and TdTomato− EC.

FIG. 13H. (Extended Data FIG. 8). Heatmap showing genes that were commonly upregulated in TdTomato+ EC from all 5 comparisons (TdTomato+ versus TdTomato− EC from P1 heart, P1 lung EC, P6 muscle, 6 week heart, 6 week muscle) and were significant (FDR<0.05) in at least two groups.

FIG. 13I. (Extended Data FIG. 8). Confirmation of the expression selected genes from FIG. 4B and FIG. 13H using quantitative RT-PCR using adult ABCG2TT muscle TdTomato+ and TdTomato− EC (n=7).

FIG. 13J. (Extended Data FIG. 8). Percentage of Sele+ cells in TdTomato+ and TdTomato− EC (PI-CD45-Ter119-CD31+) 1 day after tamoxifen injection. Data represent mean±s.d. p values, two-tailed unpaired t-test. (n=10 mice; k, heart, n=4, lung, n=6, muscle, n=5).

FIG. 13K. (Extended Data FIG. 8). Percentage of InsR+ (k) cells in TdTomato+ and TdTomato− EC (PI-CD45-Ter119-CD31+) 1 day after tamoxifen injection. Data represent mean±s.d. p values, two-tailed unpaired t-test. (j, n=10 mice; k, heart, n=4, lung, n=6, muscle, n=5).

FIG. 13L. (Extended Data FIG. 8). Heatmap of genes expression among all samples (TdTomato+ and TdTomato− EC from p1 heart, p1 lung, p6 muscle, 6 week heart, and 6 week muscle).

FIG. 13M. (Extended Data FIG. 8). GO pathway analysis between TdTomato+ VESC from neonatal and adult heart of ABCG2TT mice.

FIG. 13N. (Extended Data FIG. 8). GO pathway analysis between TdTomato+ VESC from neonatal and adult muscle (n) of ABCG2TT mice.

FIG. 13O. (Extended Data FIG. 8). Numbers of genes that were significantly (FDR<0.05) up- or down-regulated in TdTomato+ Abcg2-expressing VESC from neonatal heart, lung and muscle.

FIG. 13P. (Extended Data FIG. 8). Comparison of the expression of lung, heart and muscle tissue-specific EC angiocrine factors (AF) among neonatal heart (hrt), muscle (mus) and lung (lng) TdTomato+ Abcg2− expressing VESC.

FIG. 13Q. (Extended Data FIG. 8). Jaccard distance analysis of all neonatal heart (hrt), muscle (mus) and lung (lng) TdTomato+ Abcg2-expressing VESC samples based on the expression of lung, heart and muscle tissue-specific EC angiocrine factors. Smaller number indicates two samples were more similar to each other.

FIG. 14A. (Extended Data FIG. 9). Human VESC are labeled by ABCG2. a. Representative flow cytometry data (from 4 patients) showing the analysis of fresh human umbilical cord vein endothelial cells (HUVEC) using putative vascular endothelial stem cell (VESC) markers.

FIG. 14B. (Extended Data FIG. 9). Quantitation of the percentage of fresh human umbilical cord artery endothelial cells (HUAEC, b) that express each putative VESC markers. Data represent mean±s.d. (HUAEC, n=3; HUVEC, n=4).

FIG. 14C. (Extended Data FIG. 9). Quantitation of the percentage of fresh human umbilical cord artery endothelial cells (HUVEC (c) that express each putative VESC markers. Data represent mean±s.d. (HUAEC, n=3; HUVEC, n=4).

FIG. 14D. (Extended Data FIG. 9). Purity of magnetic activated cell sorting separated ABCG2+ HUVEC tested by flow cytometry.

FIG. 14E. (Extended Data FIG. 9). Representative pictures of EC colonies derived from ABCG2+ HUVEC (150 EC on 1 well of 6 well plate, left panel) and ABCG2− HUVEC (1,000 EC on 1 well of 6 well plate, right panel).

FIG. 14F. (Extended Data FIG. 9). Representative picture showing the perfusion of single ABCG2+ HUVEC derived EC formed blood vessel after in vivo transplantation through the colocalization of Ulex Europaeus Agglutinin I (Lectin) and CD31. Red, lectin; Cyan, CD31.

FIG. 14G. (Extended Data FIG. 9). Single ABCG2+ HUVEC derived blood vessels 2 weeks after co-transplantation with OP9 (represents 4 patients). Cyan, human CD31; Red, smooth muscle actin α (SMA).

FIG. 14H. (Extended Data FIG. 9). Representative pictures of a secondary HUVEC colony derived from single ABCG2+ HUVEC transplanted gel.

FIG. 14I (Extended Data FIG. 8) Representative picture of secondary blood vessels in host mouse derived from secondary colony EC retrieved from single ABCG2+ HUVEC primary transplanted gel. Cyan, human CD31.

FIG. 15A. (Extended Data FIG. 10). Schematic of the contribution of ABCG2-expressing vascular endothelial stem cells to vessel maintenance.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

While a number of cardiovascular diseases have been linked with abnormal resident and circulating EC colony forming ability⁴⁵⁻⁴⁷, the concept of vascular endothelial stem/progenitor cells has not been widely appreciated. Here, for the first time, we have identified a marker Abcg2 (ABCG2 in human) that labels VESC in both man and mice, and provide evidence that these cells fulfill all criteria of true stem cells that have been adapted from the definition of other lineage-specific stem cells. In our lineage tracing model using Abcg2TT mice, rare Abcg2-VESC in neonatal mice were found to significantly contribute to vessel growth/maintenance in multiple organs for up to 18 months. In adult mice, Abcg2-VESC persist and have the potential to participate in vessel maintenance and regeneration after injury. Thus, for those cardiovascular disease patients with diminished vascular EC proliferative potential, like peripheral artery disease patients⁴⁶, ABCG2 is a potential target to identify resident VESC to better define the pathophysiologic mechanisms and develop potential strategies for their treatment.

### EXAMPLES

### Material and Methods

Animals

All animal experiments were conducted in accordance with the Guidelines for the Care and Use of Laboratory Animals, and all protocols were approved by Institutional Animal Care and Use Committee of the Indiana University School of Medicine. C57BL/6 (JAX stock #000664), B6.Cg-Gt(ROSA)26Sor^{tm14(CAG-tdTomato)Hze}^{_{/J}}¹a (R26R-TdTomato, JAX stock #007914), NU/J (athymic nude, JAX stock #002019), NOD.CB17-Prkdc^{scid}/J (NOD/SCID, JAX stock #001303) were purchased from the Jackson Laboratory. Bcrp Constitutive knock out (ABCG2 knockout, #2767) were obtained from Taconic. Cryopreserved sperm of Abcg2CreERT mice^{2a} was kindly provided by Dr. Brian Sorrentino and the transgenic colony was recovered through in vitro fertilization services provided by the Indiana University School of Medicine Transgenic Mouse Core. The following primers were used for genotyping the above mentioned: Cre mice: Cre F: 5′-CGG TCG ATG CAA CGA GTG AT-3′ (SEQ. ID NO. 17); Cre R: 5′-CCA CCG TCA GTA CGT GAG AT-3′ (SEQ. ID NO. 18).

Drug Administration

Tamoxifen (Sigma-Aldrich) was suspended in sunflower seed oil (Sigma-Aldrich) at 37° C. to make 4 mg/ml solution and was stored at −20° C. until use. To induce Cre expression in ABCG2CreERT mice, 50 mg/kg tamoxifen was injected into the animals intra-peritoneally (i.p.) at appropriate time points.

Patient Samples

Human umbilical cord samples were collected from scheduled term newborn Cesarean deliveries. Since no identifying information was collected on the patients, use of the umbilical cord tissue was deemed surgical waste material and not human research by the Indiana University Institutional Review Board. Human saphenous vein samples were also collected as leftover surgical waste tissue from patients undergoing coronary bypass surgery and delivered to the authors in unlabeled tubes lacking any patient identifying information.

Cell Collection

To collect cells from mouse lung, muscle, skin and heart, tissues were dissected from euthanized mice and were minced with blades. Samples were digested with 0.25% collagenase I (Stem Cell Technologies) at 37° C. for 30 minutes. After digestion, the samples were re-suspended in medium, pipetted thoroughly, and passed through 70 μm cell strainers to removed cell clumps. To collect mouse bone marrow cells, tibias and femurs were dissected and cleaned with scissors to remove remaining muscle tissues. Then the bones were crushed with a pestle in a mortar before digesting and straining as above.

To collect human umbilical cord artery or vein EC, vessels were flushed by PBS for 3 times. Then one end of the vessel was clamped and liberase solution (Roche, 500 μl stock solution diluted with 24.5 ml PBS) was infused into the vessel through the open end before it was clamped. The liberase infused vessels were incubated at 37° C. for 14 minutes to release EC from the basement membrane. Finally, the solution containing digested EC was flushed into 50 ml tubes for centrifugation.

Magnetic Activated Cell Sorting (MACS)

For murine CD45⁺ cell depletion, blood cells or digested tissue cells were re-suspended in sorting buffer (PBS plus 1% FBS and 5 mM EDTA) and stained with biotin conjugated anti mouse CD45 antibody (BD Biosciences, clone 30-F11). CD45⁺ hematopoietic cells were subsequently depleted using Stemcell technologies EasySep™ Mouse Streptavidin RapidSpheres™ Isolation Kit. For CD31 positive sorting, cells were stained with biotin conjugated anti mouse CD31 antibody (Miltenyl Biotec, clone 390) and CD31⁺ EC were isolated using EasySep™ Biotin Positive Selection Kit (Stemcell technologies). ABCG2⁺ cells from human umbilical cord vein EC were sorted using biotin-anti human ABCG2 antibody (eBioscience, clone 5D3) and EasySep™ Biotin Positive Selection Kit (Stemcell technologies). After sorting, the purity of sorted cells, the percentage of CD31⁺CD45⁻ EC were measured by flow cytometry (see below).

Flow Cytometry

The following anti-mouse antibodies conjugated with different fluorochrome were used for flow cytometry sorting and analysis: CD31 (clone 390), CD45 (30-F11), Ter119 (TER-119), ProcR (eBio1560), c-Kit (2B8), selectin E (P2H3) (all above antibodies were purchased from eBioscience), CD157 (BioLegend, clone BP-3), and Insulin receptor (R&D Systems, FAB1544G). For human cell flow cytometry analysis, the following anti-human antibodies were used: CD31 (BD Pharmingen or eBioscience, clone WM59), CD45 (eBioscience or BioLegend, clone 2D1), CD34 (eBioscience, clone 4H11), ABCG2 (eBioscience, clone 5D3), PROCR (eBioscience, clone RCR-227), CD157 (eBiocience, clone eBioSY11B5), and CKIT (BioLegend, clone 104D2). 1:1000 propidium iodide (PI, Sigma-Aldrich) was added to sorting buffer before analysis to distinguish live from dead cells. Cell analysis and sorting were performed on LSR 4, LSRII, FACSCantol I, FACSAria, SORPAria flow cytometers (BD Biosciences). FlowJo software was used to analyze flow cytometry data. For generating Venn diagrams to compare different murine endothelial stem cell markers, Vennerable r package (http://r-forge.r-project.org/projects/vennerable) was used.

For SP staining, cells were stained for surface antigens first and then 1 million of stained MNC were re-suspended in 1 ml of SP buffer (DMEM with 2% FBS and 1 mM HEPES). Next 5 μg/ml Hoechst 33342 (Sigma-Aldrich) was added to the cell suspension and incubated at 37° C. for 90 minutes with or without 50 μmol/l Verapamil (Sigma-Aldrich). Finally, the cells were re-suspended in sorting buffer before they were analyzed/sorted with SORPAria flow cytometer with an ultra-violet laser. Culture of Endothelial Colonies For murine EC culture, OP9 stromal cells were maintained in OP9 medium (alpha-MEM medium [Gibco], with 20% FBS [Hyclone], and 0.5% penicillin/streptomycin [Gibco]). To culture endothelial colonies, isolated endothelial cells or peripheral blood MNC were re-suspended in EC culture medium (alpha-MEM with 10% FBS [Hyclone], 5×10⁻⁵ M β-mercaptoethanol [Sigma-Aldrich] and 0.5% penicillin/streptomycin [Gibco]). After 24 hours, non-adherent cells were removed by changing spent to fresh medium. Medium was changed every 3 days afterwards until use. For human EC culture, the cells were re-suspended in complete EGM2 medium (Endothelial Basal Medium-2 [EBM-2, Lonza] with 10% FBS [Hyclone]) and re-plated on 0.1% type 1 rat tail collagen (BD Biosciences) coated tissue culture plates.

Surgeries

For EC collagen gel transplantation assay, cells were re-suspended in 250 ul 200 pa pig skin type I collagen gel (Geniphys, Standarized Oligomer Polymerization Kit) plus 10% human platelet lysate (Cook) on ice. When murine EC were tested, 50 μg/ml murine VEGF (Peprotech) and 100 μg/ml murine FGF (Peprotech) were added to the gels. Each cellularized gels were transferred into 1 well on 48 well plate and incubated at 37° C. to polymerize for 30 minutes. Next the cellularized gels were transplanted into the flanks of 6-12 weeks old NOD/SCID mice as previously described^{3a}. The gels were retrieved from the animals at various time points between 14 days and 10 months.

Hind limb ischemia experiments were operated as previously described^{3a}. Briefly, after the 6-8 weeks old athymic nude mice were anesthetized with isoflurane, a skin incision on their left inner thigh was made. The distal and proximal ends of the femoral artery were ligated and the portion of femoral artery between these two ligatures was excised. After the excision, 200 μl cell suspension in PBS or control PBS, were injected into 4 sites of the gracilis muscle. Then the incisions were sutured closed and a Laser Doppler imager (Moor Instruments) was used to measure the blood flow in the injured and control legs (day 0) and every 7 days post-treatment until 6 weeks. The mean perfusion values from each leg was measured and recorded using instructions as supplied in the Moor software.

Cell Culture Immunohistochemistry and Immunofluorescent Staining

For immunohistochemistry staining of endothelial colonies on OP9 co-culture plates, the cultures were fixed with 4% paraformaldehyde (PFA) for 30 minutes at RT. After washing, the samples were blocked with 2% skim milk (Sigma-Aldrich) in 0.1% triton (Sigma-Aldrich) PBS solution (PBSMT solution) for 30 minutes at RT and then stained with 1:100 rat anti mouse CD31 (BD Pharmingen, clone MEC 13.3) or rat anti mouse Flk1 (BD Pharmingen, clone Avas 12α1) antibody in PBSMT at RT for 2 hours or at 4° C. overnight. Next the plates were stained with 1:200 alkaline phosphatase conjugated donkey anti rat IgG secondary antibody (Jackson ImmunoResearch) in PBSMT at RT for 2 hours or at 4° C. overnight. Finally the colonies were visualized using a Leica™ DM IL microscope with a SPOT RT3 camera (Spot Imaging).

For immunofluorescent staining of cultured cells, fixed cultures were blocked with 10% goat serum in 0.5% triton PBS solution (blocking solution) and sequentially stained with primary antibodies (1:100 rat anti mouse CD31) and secondary antibody (1:200 Alexa Fluor 488 conjugated goat anti rat IgG [Cell Signaling Technology]) in blocking buffer.

Tissue Immunofluorescent Staining

To visualize TdTomato+ vessels in freshly collected muscle or collagen gel samples after transplantation, a Leica™ mz9.5 stereomicroscope with LEJ eqb 100 isolated lamp power supply was used. To detect the perfusion of implanted vasculature that had inosculated with host vessels, 100 ul fluorescein conjugated isolection B4 (Vector Laboratories, for mice vessels) or 100 ul fluorescein labeled Ulex Europaeus Agglutinin I (UEA I, Vector Laboratories, for human vessels) were intravascular injected into the mice 30 minutes prior to euthanization and sample collection. To take confocal images of tissues or transplanted gels, the samples were collected and fixed in 4% PFA at 4° C. overnight, rinsed in 30% sucrose at 4° C. overnight, and then mounted in O.C.T. compound (Fisher Scientific) on dry ice. The tissue blocks were cut into 10-30 sections μm sections using a Leica CM3050s cryostat and mounted on Superfrost Plus Gold microscope glass slides (Thermo Fisher Scientific). After blocking with blocking buffer at RT for 1 hour, the slides were then stained with different unconjugated primary anti bodies include: rat anti mouse CD31 (BD Pharmingen, clone MEC 13.3, 1:100), rabbit anti ERG (Abcam, clone EPR3864. 1:100), or mouse anti human ABCG2 (Abcam, clone BXP-21. 1:50), at 4° C. overnight. Then 1:200 Alexa Fluor 488, 555, or 647 conjugated goat anti rat, anti-rabbit, or anti mouse IgG antibodies (Cell Signaling Technology) were used for secondary staining at 4° C. overnight. For some staining, the following conjugated antibodies were used: Alexa Fluor 647 conjugated mouse anti human CD31 (BD Pharmingen, Clone WM59. 1:50), Alexa Fluor 488 or 594 conjugated mouse anti smooth muscle actin α (eBioscience, clone 1A4. 1:100). After staining, the samples were mounted with ProLong™ Gold Antifade Mountant with DAPI (Molecular Probes) and Z-stack confocal images were taken on an Olympus FV1000 microscope.

For whole mount tissue deep imaging, neonatal tissues were fixed in 4% PFA for 1 hour at RT. Older mice were perfused with 10 mL PBS followed by 10 mL 4% PFA at 3 mL/min. Dissected tissues were further fixed in 4% PFA for 1 hour at RT, rinsed twice with 1×PBS. Samples were cleared for 24-48 hours in PBS with 10% Triton X-100 (w/v) and 5% N,N,N′,N′-Tetrakis (2-Hydroxypropyl)ethylenediamine (Sigma) at 37° C. with mixing, followed by two PBS washes 1 hour each. Nonspecific binding was blocked by a 3 hour incubation in PBS containing 0.1% Triton X-100 and 10% normal goat serum (PBSTS) at RT. Samples were then incubated with primary antibodies (1:100 Alpha-Smooth Muscle Actin Monoclonal Antibody (1A4), Alexa Fluor 488 (ThermoFisher cat #53-9760-80) and 1:100 CD31 (clone 2H8) (ThermoFisher cat # MA3105) diluted in PBSTS overnight at RT. In the morning slides were then washed 3 times with PBST. Samples were incubated in secondary antibody (1:200 Alexa Fluor 647 labeled goat anti-Armenian hamster (Jackson ImmunoResearch cat #127-605-160) diluted in PBST) overnight at RT, then washed 3 times with PBST 2 hours each. Refractive index matching was accomplished by overnight incubation in RIMS^{4a} at 37° C.

Image acquisition was performed using a Leica SP8 Confocal Microscope using a 20×NA 0.75 multi-immersion objective at 1-μm intervals. Large scale confocal imaging of overlapping volumes was performed with an automated stage and stitched using Leica LAS X software (Germany). 3D tissue cytometry was performed on image volumes using VTEA^{5a}.

All fluorescent pictures were processed using ImageJ software to produce merged images. 3D reconstruction of CD31+ and TdTomato+ vessels in tissues or gels was performed using Imaris software. The volume of blood vessels was calculated from the images by Imaris software using the “Surface” function.

RNA Isolation and RNAseq

Cells were selected using a SorpAria flow cytometer as above. Total RNA was extracted using a Qiagen RNeasy micro kit, followed by standard adaptor ligation and library construction steps. Illumina TruSeq RNA Access Library Prep Kit was used to prepare dual-indexed strand-specific cDNA library. Ribosomal RNAs were depleted using polyA selection. Sequencing was performed at the Indiana University Center for Medical Genomics Core with 2×75 bp paired-end configuration on HiSeq4000 using HiSeq 3000/4000 PE SBS Kit. The sequenced data were mapped to the mm10 mouse genome using STAR RNA-seq aligner. Uniquely mapped sequencing reads were assigned to mm10 refGene genes using feature Counts. Differential expression analysis was performed using exactTest and glmLRT (edgeR). P values were adjusted with FDR method as indicated. All plots were generated in R software 3.4.3 using heatmap3, levelplot and VennDiagram.

Quantitative PCR

RNA from each sample was extracted using RNeasy Micro kit (Qiagen). Reverse transcription was done using Omniscript RT Kit (Qiagen). For validation of RNAseq data, Taqman Fast Advanced Master Mix, primers and probes were purchased from Thermo Fisher Scientific. Beta-actin, Gapdh, and 18S rRNA were used as endogenous control. Quantitative PCR was performed on Applied Biosystems® 7500 Real-Time PCR System. For ATP binding cassette transporters, quantitative PCR was performed on Applied Biosystems® 7500 Real-Time PCR System with FastStart Universal SYBR Green Master (Roach) in triplicate. Beta-actin was used as reference gene to calculate transcript abundance of each target gene. The expression level folds change between sample genes and reference genes were calculated by 7500 software. The following primers were used:

ABCG2F:
(SEQ. ID NO. 1)
5′-CCATAGCCACAGGCCAAAGT-3′ (Sequence from^{6a}).
ABCG2R:
(SEQ. ID NO. 2)
5′-GGGCCACATGATTCTTCCAC-3′ (ref The ABC
transporter Bcrp1/ABCG2 is expressed in a wide
variety of stem cells and is a molecular determi-
nant of the side- population phenotype)(Sequence
from^{6a}).
ABCB1bF:
(SEQ. ID NO. 3)
5′-TGATCATCAGCAACAGCAGTC-3′ (Sequence from^{6a}).
ABCB1bR:
(SEQ. ID NO. 4)
5′-TGAAACCTGGATGTAGGCAAC-3′ (Sequence from^{6a}).
ABCB2F:
(SEQ. ID NO. 5)
5′-CTCTTGCCTTGGGGAAATG-3′ (Sequence from^{6a}).
ABCB2R:
(SEQ. ID NO. 6)
5′-CTGTGCTGGCTATGGTGAGA-3′ (Sequence from^{6a}).
ABCC7F:
(SEQ. ID NO. 7)
5′-GACACTTTGCTTGCCCTGAG-3′ (Sequence from^{6a}).
ABCC7R:
(SEQ. ID NO. 8)
5′-AAGAATCCCACCTGCTTTCA-3′ (Sequence from^{6a}).
ABCA5F:
(SEQ. ID NO. 9)
5′-TTCTATGTCCTCCTGGCTGTG-3′ (Sequence from^{6a}).
ABCA5R:
(SEQ. ID NO. 10)
5′-TGACCAATACGATGGCTTCA-3′ (Sequence from^{6a}).
ABCA3F:
(SEQ. ID NO. 11)
5′-TTATGCCCTCCTACTGGTGTG-3′ (Sequence from^{6a}).
ABCA3R:
(SEQ. ID NO. 12)
5′-CTTGTCCTTATTGCCCACTTG-3′ (Sequence from^{6a}).
ABCB1aF:
(SEQ. ID NO. 13)
5′-CCAGCAGTCAGTGTGCTTACA-3′.
ABCB1aR:
(SEQ. ID NO. 14)
5′-GCCACTCCATGGATAATAGCA-3′ (From ^{7a}).
Beta-actinF:
(SEQ. ID NO. 15)
5′- TCCTGTGGCATCCATGAAACT-3′.
Beta-actinR:
(SEQ. ID NO. 16)
5′- GAAGCACTTGCGGTGCACGAT-3′(From ^{8a}).

Western Blot Analysis.

Flash-frozen tissues were crushed with a pestle in a mortar. The crushed tissues were washed twice with ice-cold phosphate-buffered saline and lysed on ice in RIPA buffer (Sigma-Aldrich) supplemented with protease inhibitor (Roche). Cell lysates were sonicated and centrifuged at 13,200 rpm for 10 min; boiled with LDS sample buffer (ThermoFisher Scientific); separated by NuPAGE gel (ThermoFisher Scientific); transferred electrophoretically to a PVDF (EMD Millipore); and immunoblotted with ABCG2 antibody (Abcam, clone BXP-21), and GAPDH antibody (Cell Signaling Technology), followed by incubation with HRP-conjugated secondary antibodies (Cell Signaling Technology). The blots were developed using the enhanced chemiluminescence technique with HRP substrate peroxide Solution (EMD Millipore).

Statistical Analysis.

Unless otherwise mentioned, all data are presented as mean±standard deviation and unpaired two-tailed Student's t-test was used to determine significance. Any p value >0.05, was considered non-significant (n.s.). *: p<0.05, **: p<0.01, ***: p<0.001, ****: p<0.0001. All statistical analyses were performed using Graphpad Prism or Microsoft Excel software. In all figures unless otherwise mentioned, n represent biological replicates (the number of mice or number of human patients that were used in each experiment) and the numbers were provided in figure legend for each experiment. No statistical method was used to pre-determine sample size.

### Example 1

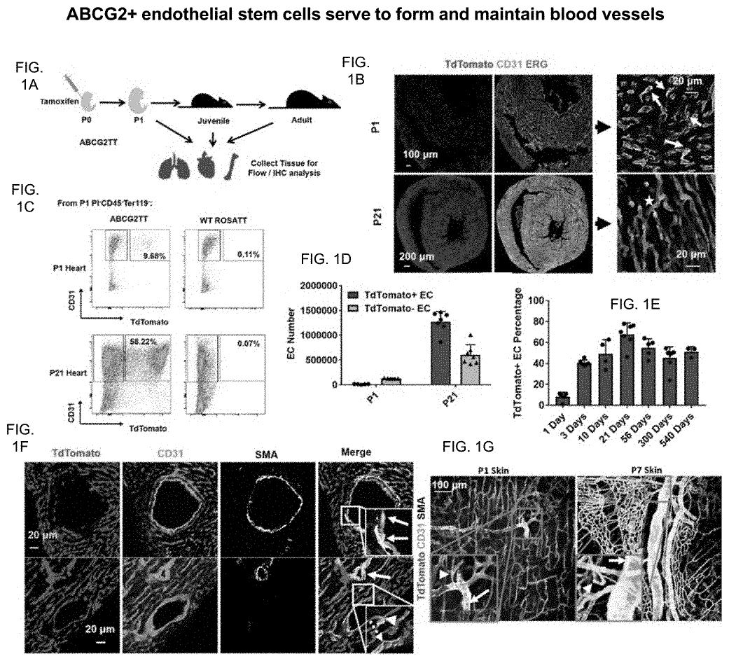

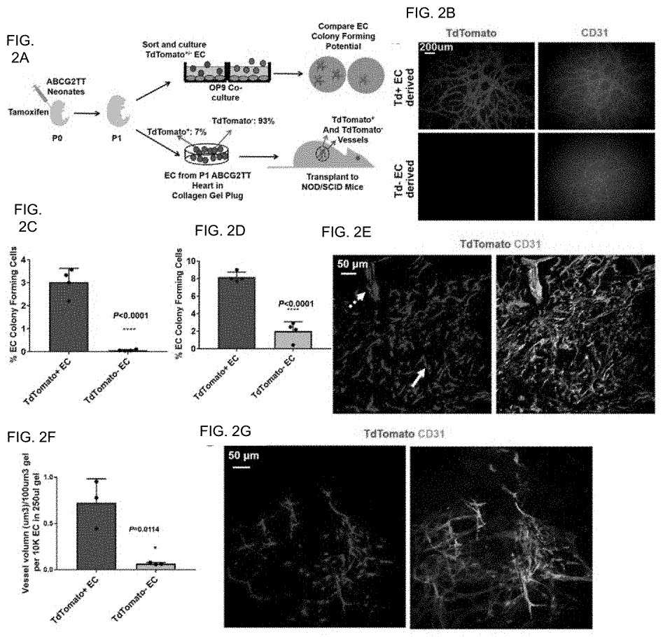

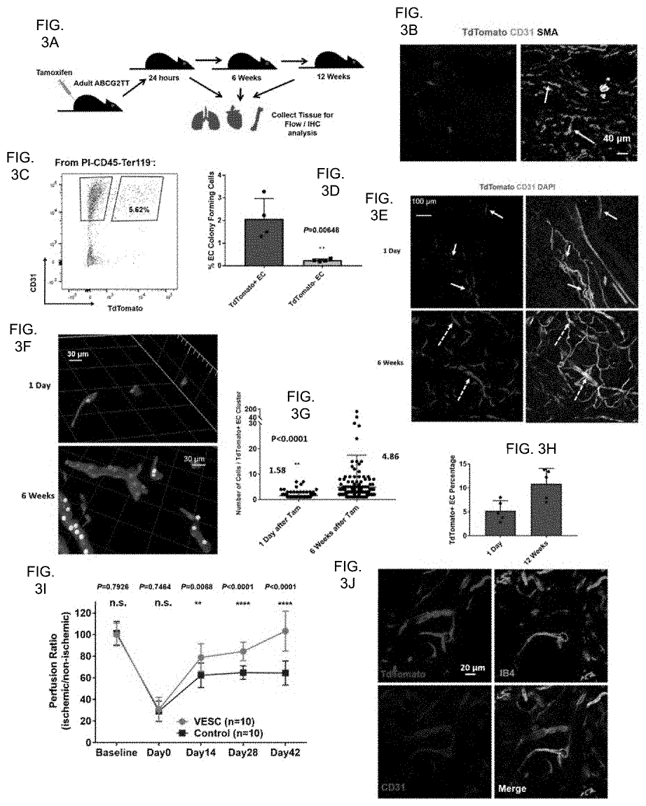

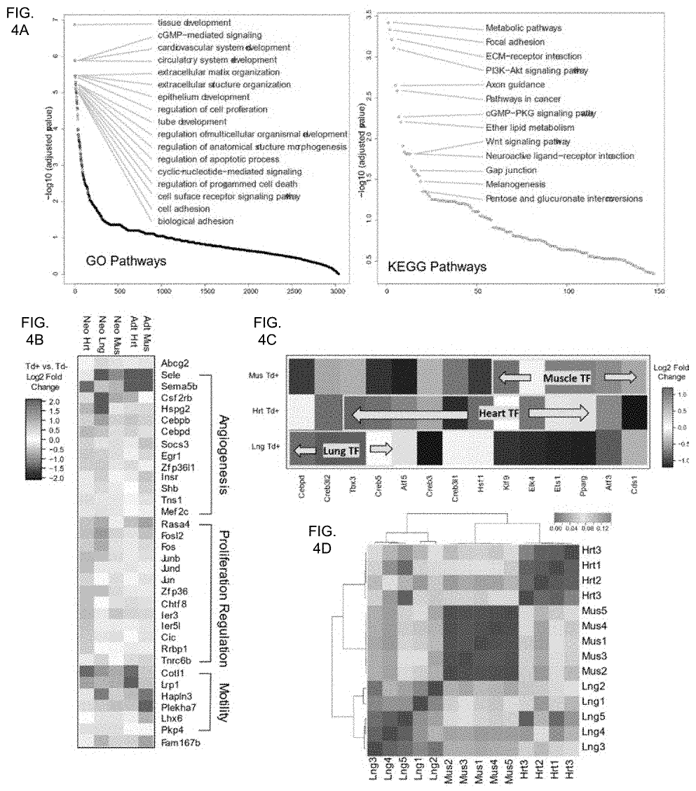

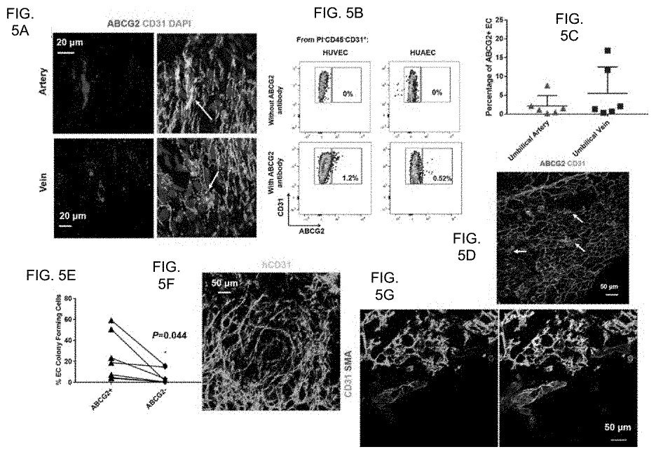

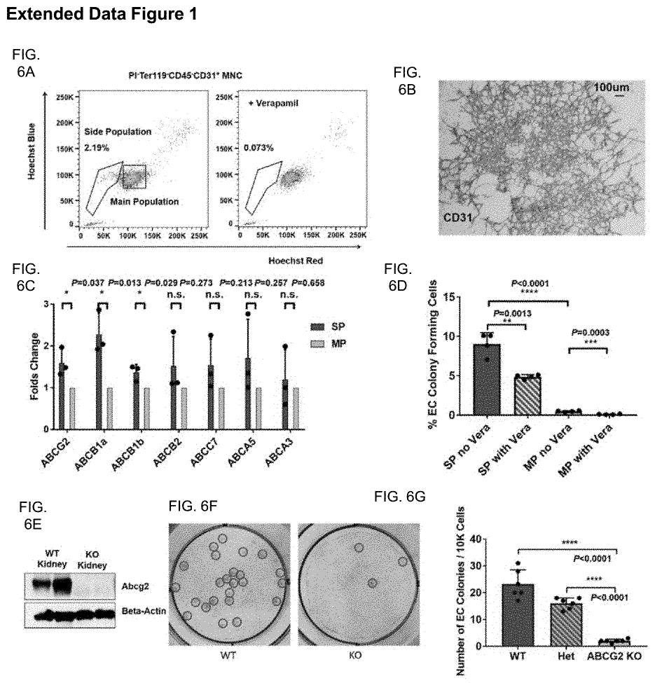

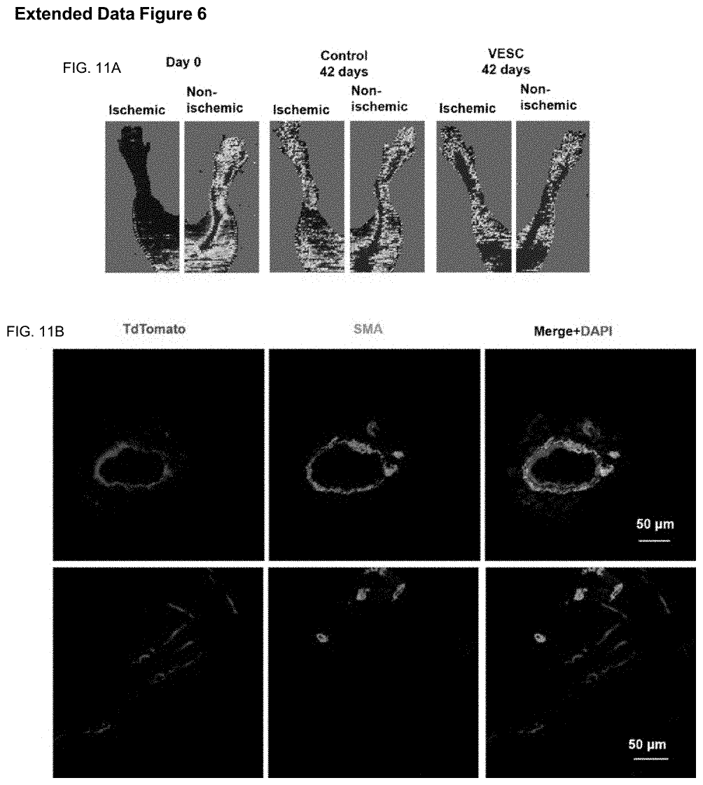

Abcg2-expressing endothelial stem cells contribute to vessel development in vivo. The SP phenotype (FIG. 6A, Extended Data FIG. 1a) labels stem/progenitor cells in multiple lineages¹⁶ include some immature vascular EC with in vitro colony forming potential (FIG. 6B, Extended Data FIG. 1b)^{4,9}. Since various ATP binding cassette (ABC) family transporters are crucial for the SP phenotype, we compared the level of transcript expression of several ABC family members in the endothelial SP with level of expression in the main population (MP) that do not possess Hoechst 33342 efflux function. The primary murine lung EC SP fraction highly expressed Abcg2 (Bcrp2) mRNA, as well as, two other members of the ABC family transporters, Abcb1a (Mdr1a) and Abcb1b (Mdr1b) (FIG. 6B, Extended Data FIG. 1c). Since Abcg2 is known to be the molecular determinant of the SP in other tissue stem cell lineages^{17,18}, we examined the role of Abcg2 in the emergence and maintenance of EC stem/progenitor cells. Indeed, both in vitro inhibition of Abcg2 function by addition of the inhibitor verapamil or loss of Abcg2 expression in Abcg2 knockout mice resulted in a significant depletion of endothelial colony-forming cells (ECFC, FIG. 6D-6G, Extended Data FIG. 1d-1g). These results are consistent with previously published data that the depletion of Abcg2 affects the emergence, maintenance, and survival of numerous tissue stem/progenitor cells and diminishes the ability of EC to repair and replenish damaged blood vessels after acute cardiovascular tissue injuries²³⁻²⁶.

### Example 2

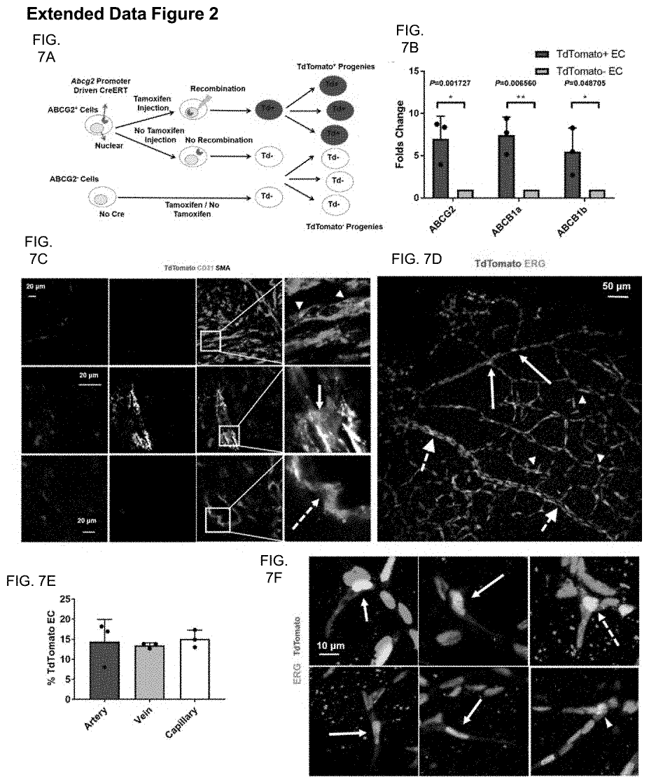

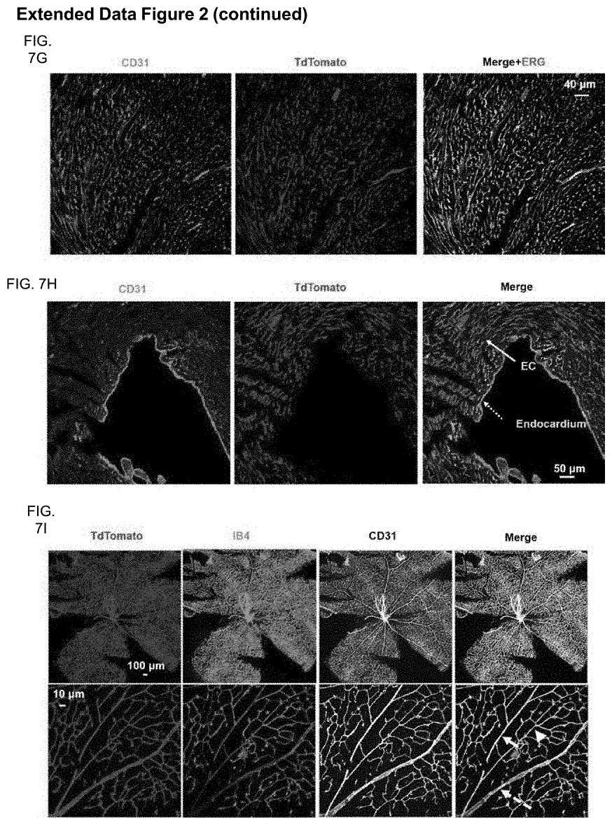

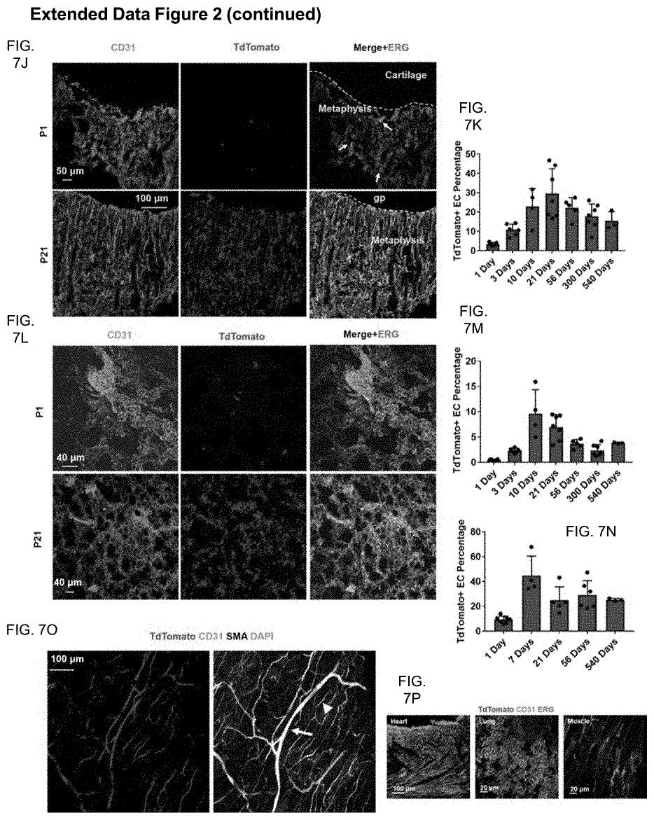

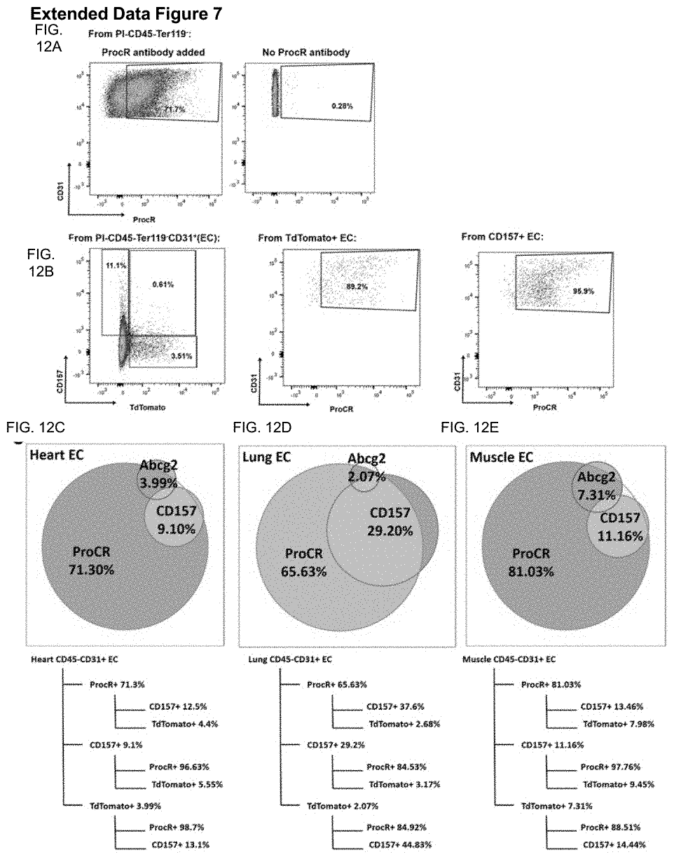

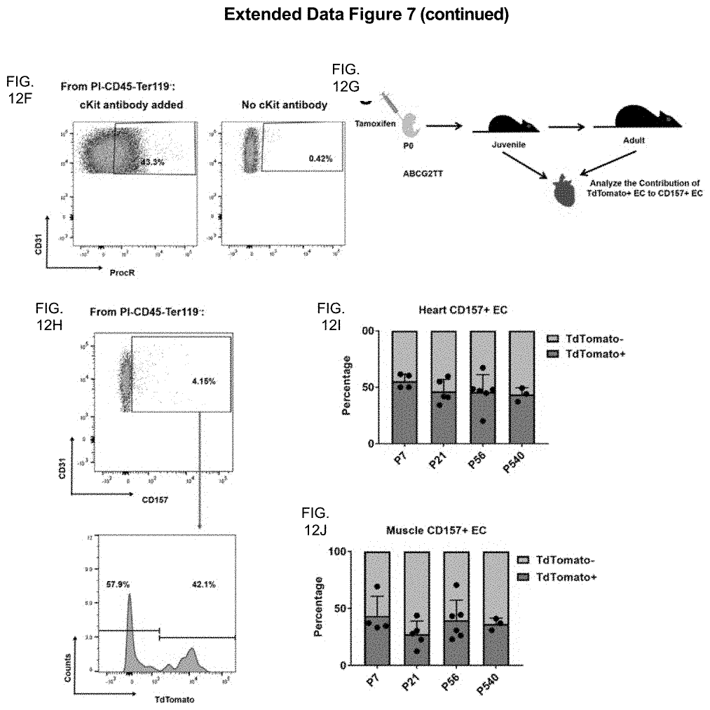

Abcg2-expressing EC contribute to vessel growth in vivo during development. Because Abcg2 expression was important for the maintenance of vascular ECFC proliferative potential, we reasoned that the expression of Abcg2 may be useful to identify putative VESC upstream of the ECFC in the vascular endothelium. By breeding mice transgenic for a tamoxifen inducible Abcg2 promoter driven Cre recombinase (Abcg2CreERT2²⁷) with ROSATdTomato mice, we generated ABCG2TT mice to study the contribution of Abcg2-expressing EC (Abcg2-EC) to the development and maintenance of blood vessels in the murine system (FIG. 1a, FIG. 7A, Extended Data FIG. 2a). First, to survey the distribution of Abcg2-expressing EC during development, ABCG2TT pups were injected with 50 mg/kg body weight tamoxifen on postnatal day 0 (P0). After 24 hours, a small fraction of EC (CD31+) in multiple tissues of P1 pups were co-labeled with TdTomato (8.1±4.1% in heart EC, 0.5±0.09% in lung EC, 3.4±0.9% in bone marrow EC, n=5, FIG. 1c, FIG. 7k, 7M, Extended Data FIG. 2k, 2m), while tamoxifen injected wild type ROSATdTomato mice (TT) displayed no TdTomato⁺ cells (FIG. 1c). P1 TdTomato+ EC expressed higher Abcg2 transcripts (FIG. 7B, Extended Data FIG. 2b), and the majority of TdTomato+ EC were single cells and not grouped as cell clusters (FIG. 1b), suggesting at this time, most TdTomato⁺ EC were Abcg2-expressing EC stem/progenitor precursors, and not their mature progeny.

TdTomato⁺ Abcg2-EC could be found in arteries (covered by a thick smooth muscle layer [strong smooth muscle actin α]^{28,29}), veins (diameter >20 μm, covered by a thin smooth muscle layer [weak or no smooth muscle actin α]^{28,29}) and capillaries (diameter <10 μm²⁸) (FIG. 7C, Extended Data FIG. 2c). Since the murine retinal vasculature fully develops within the first 10 days of life, we investigated the distribution of Abcg2− EC in nascent retinal arteries, veins and capillaries. Tamoxifen was injected at P3 and pups analyzed at P4, a time when arteries and veins are first morphologically identifiable^{30,31}. Similar to other developing tissues, P4 retinal vessels displayed TdTomato⁺ EC in newly differentiated arteries (14.4±5.6%), veins (13.5±0.7%), and capillaries (15.1±2.2%) (n=3, FIG. 7D, 7E, Extended Data FIG. 2d, 2e). Additionally, TdTomato⁺ EC were identified as tip, stalk, and phalanx cells in the growing retinal vascular beds. Surprisingly, nearly all retinal tip cells at this stage expressed TdTomato, suggesting a predilection for Abcg2-EC as sites for angiogenic tip emergence (FIG. 7F, Extended Data FIG. 20. To investigate the contribution of neonatal Abcg2-EC to the growth of blood vessels during postnatal development, 37 ABCG2TT mice that were injected with Tamoxifen at P0 were analyzed at various developmental stages for up to 18 months (FIG. 1a).

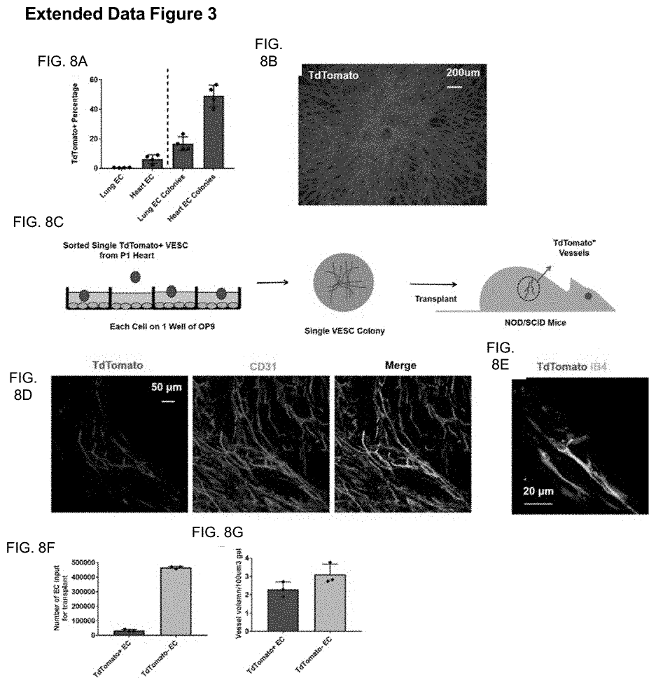

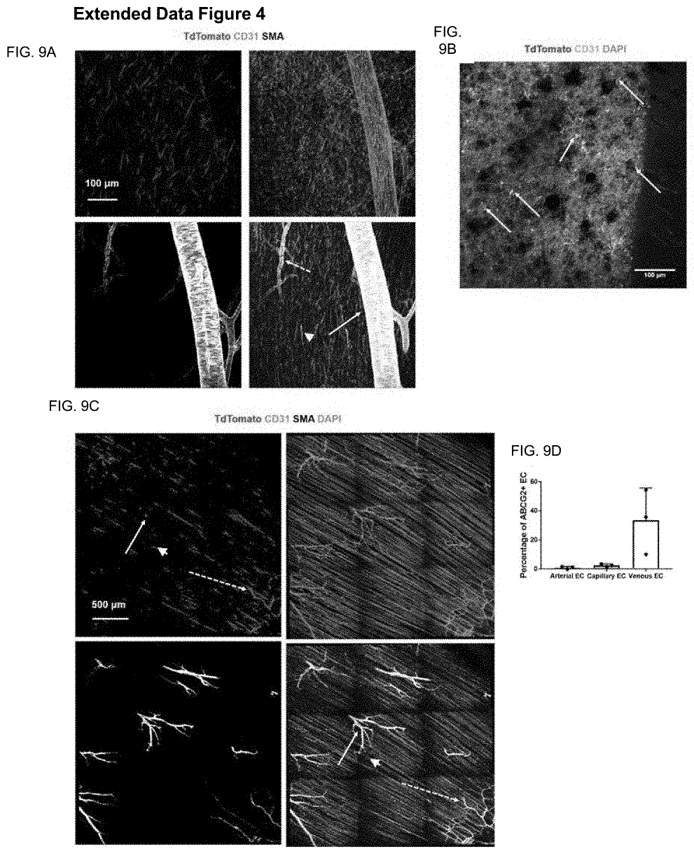

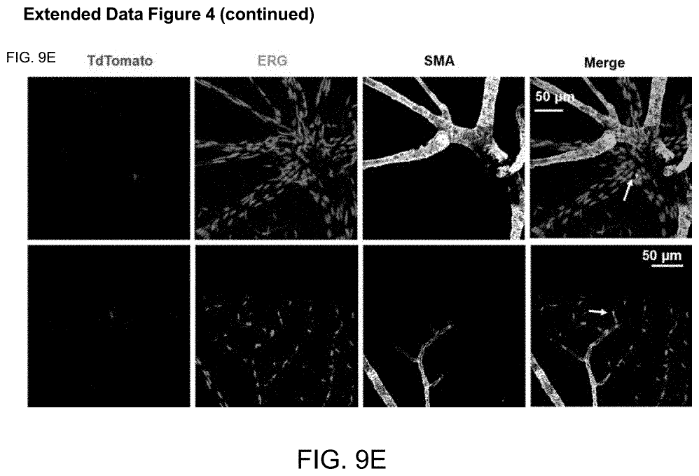

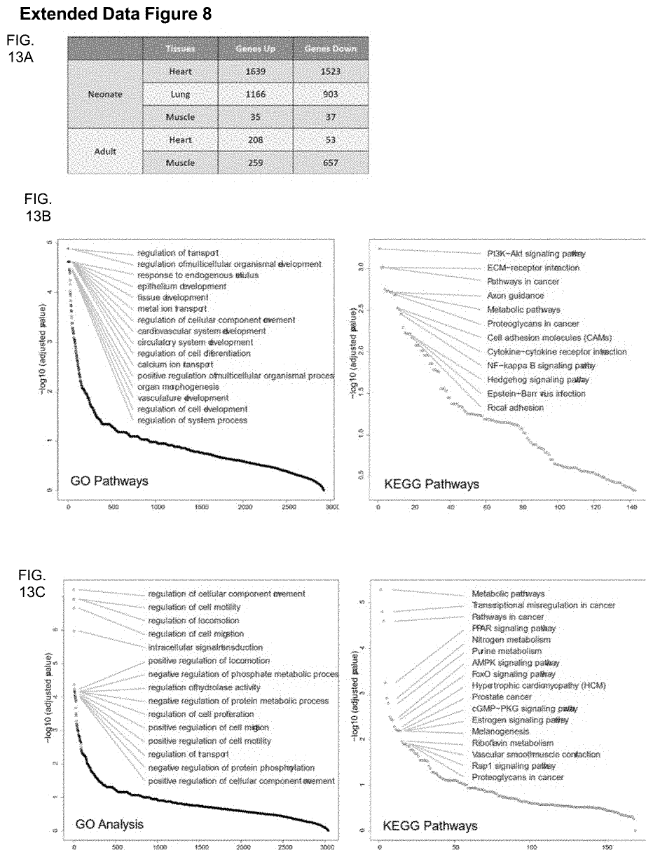

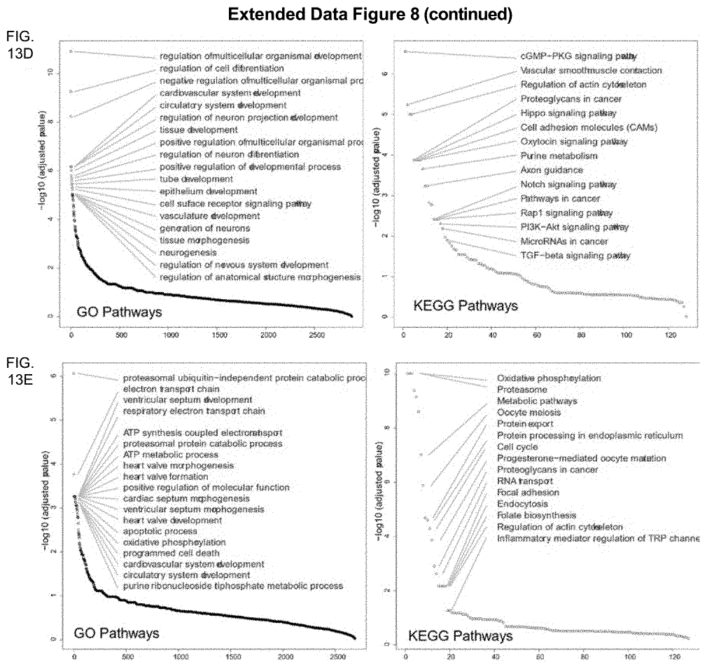

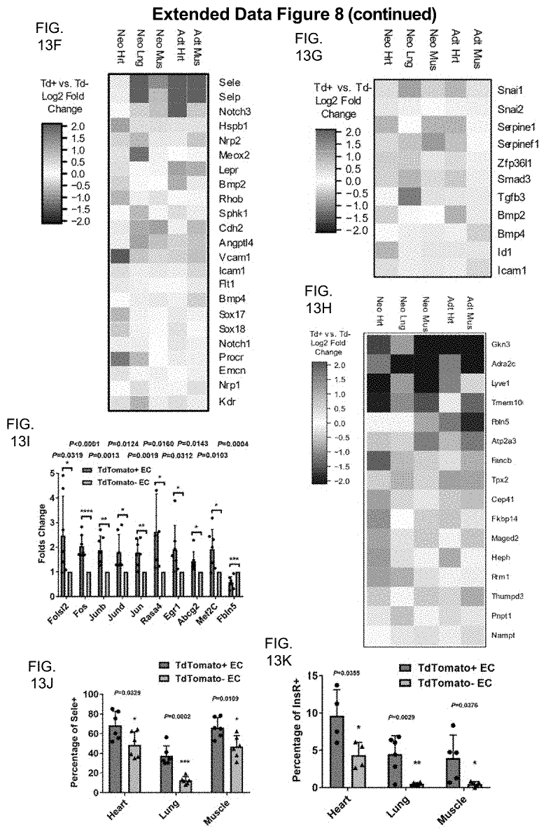

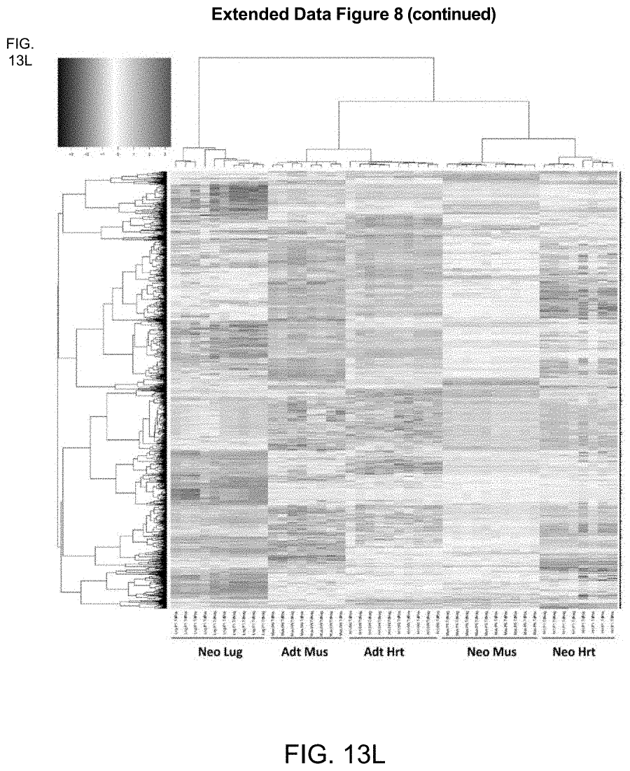

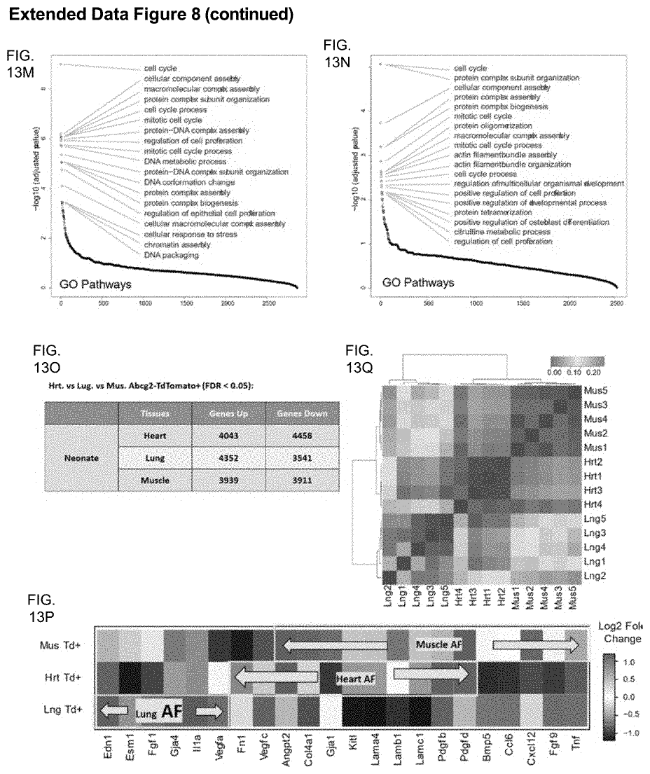

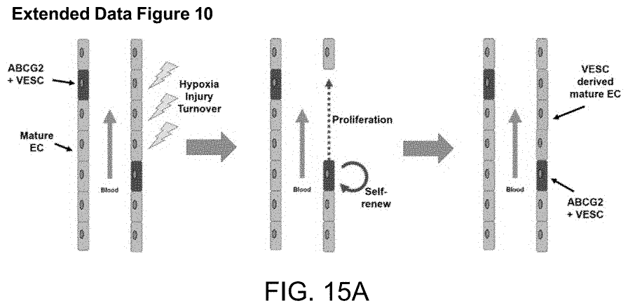

Remarkably, TdTomato⁺ Abcg2-EC, which represent only 8.1±4.1% of total EC in the heart at P1 (FIG. 1b-d), quickly expanded and contributed to 67.7±11.0% of EC in the heart of 3-week-old mice (FIG. 1d-e), including contributions to arterial, venous and capillary EC (FIG. 10. From P1 to P21, cardiac TdTomato+ expanded 114.21-fold (n=7), which was 24 times higher than TdTomato− EC (4.81-fold, FIG. 4d), suggesting that Abcg2-EC represent VESC with more pronounced clonal expansion capacity. In adult animals, progeny of P0 labeled heart Abcg2-VESC were sustained for up to 540 days after labeling (45.5±10.3%, n=3, FIG. 1e, FIG. 7G, Extended Data FIG. 2g). Interestingly, no endocardial cells were found to be derived from TdTomato⁺ VESC (FIG. 8H, Extended Data FIG. 3h). Similarly, persistent contributions (up to 18 months) from the VESC labeled at P0 were identified in retina, bone, lung, skeletal muscle, skin (FIG. 1g, FIG. 7I-7O, Extended Data FIG. 2i-o), although the degree of contribution varied among the organs. Wild type TT mice without the Abcg2-Cre transgene showed no TdTomato+ EC at P540 (FIG. 7P, Extended Data FIG. 2p). These results demonstrate that Abcg2-VESC contribute long term to mature progeny in the entire systemic vasculature (arteries, veins, capillaries) in multiple tissues during normal murine growth and development. Interestingly, in each tissue, the percentage of TdTomato+ EC reached a peak around weaning, but then were maintained at a relatively lower but steady state level in adult mice (FIG. 1e, FIG. 7K, 7M, 7N, Extended Data FIG. 2k, 2m, 2n). Thus, while some Abcg2-VESC derived EC may regress during vessel pruning and remodeling³², maintenance of a long term contribution to vessel endothelium reflects marking of resident VESC.

### Example 3

Abcg2-VESC Possess EC Colony Forming Potential and In Vivo Vessel Forming Potential.

Because the progeny of P0 labeled Abcg2-VESC significantly contributed to the development of murine blood vessels, we confirmed evidence for their stem cell features. To collect only Abcg2-VESC but not their progeny, we administrated one dose of tamoxifen to P0 animals and collected TdTomato⁺ and TdTomato⁻ EC after 24 hours from P1 heart and lung vessels by flow cytometry (FIG. 2a). After TdTomato⁺ and TdTomato⁻ EC were co-cultured over a monolayer of OP9 stromal cells (FIG. 2a) for 10 days, TdTomato⁺ EC displayed significantly greater ECFC potential than TdTomato⁻ EC (FIG. 2b-d). Although at P1 only a small fraction of EC were TdTomato⁺ (FIG. 8A, Extended Data FIG. 3a), 49.2±7.4% of ECFC colonies derived from heart and 16.7±4.7% from lung were TdTomato⁺ (n=4 mice, FIG. 8A, Extended Data FIG. 3a). Next, we sorted single P1 heart TdTomato⁺ EC and plated them on OP9 cells to grow single colonies of Abcg2− VESC derived progeny. After 14 days, we suspended the clonally-derived cells in type 1 collagen gels, and implanted the plugs into the hypoxic subcutaneous space of host syngeneic mice (FIG. 8C, Extended Data FIG. 3c). After 2 weeks, TdTomato+ EC derived from each of 6 individual clones gave rise to perfused TdTomato⁺ vessels in vivo (FIG. 8D-8E, Extended Data FIG. 3d-e), demonstrating their robust clonal in vivo blood vessel forming potential.

To compare the vessel forming potential of TdTomato⁺ Abcg2-VESC with mature TdTomato⁻ EC, we collected EC from P1 heart and transplanted these cells suspended in collagen gel plugs into recipient mice at a ratio of 1 TdTomato⁺ EC for every 11 TdTomato⁻ EC (FIG. 2a). Two weeks later, robust donor Abcg2-VESC derived TdTomato⁺ vessels were formed in all implanted gels (FIG. 2e). Those TdTomato⁺ vessels were primarily capillaries but also contributed to the macrovasculature (>50 microns, FIG. 2e) in every gel examined (n=3). Importantly, in the retrieved gels, TdTomato⁺ vessels represented 42.4±2.7% of the total vessel volume (n=3 mice, FIG. 2e, 2f, FIG. 8G, Extended FIG. 3g), although only 6.7±1.6% of the input EC were TdTomato⁺ (n=3 mice, FIG. 8F, Extended Data FIG. 3f). Thus, the in vivo vessel-forming potential of TdTomato⁺ Abcg2− VESC was 10.8±3.2 fold higher than TdTomato⁻ EC (FIG. 2f). Additionally, after the gels were digested and re-plated on OP9 stromal cells, TdTomato⁺ EC from the primary vessels formed secondary colonies that could form secondary vessels in subcutaneous implants of host mice (n=6, FIG. 2g). These data indicate that Abcg2-EC in developing mice represent VESC that display clonal proliferative potential in vitro and in vivo, possess greater vasculogenic potential than mature EC not expressing Abcg2, give rise to capillary and macrovasculature components that inosculate with the host, and display self-renewal potential in giving rise to primary and secondary blood vasculature in vivo.

### Example 4

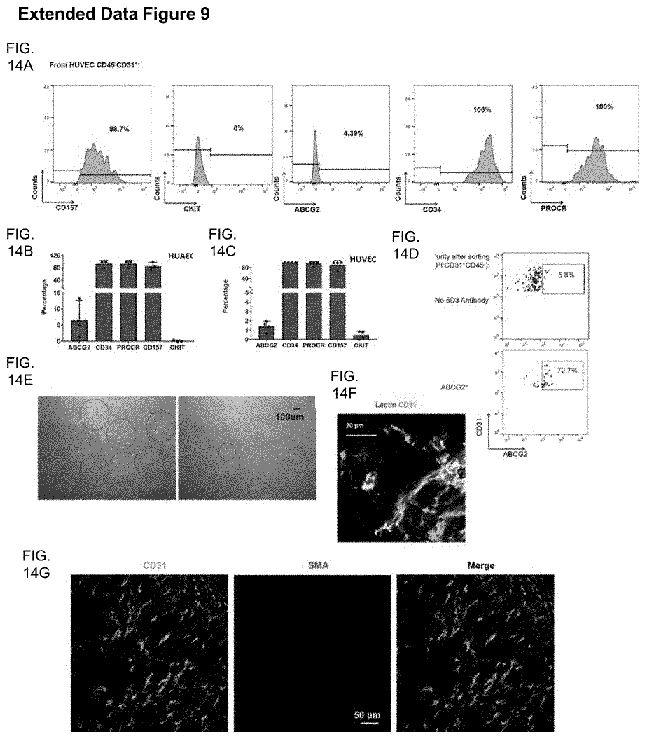

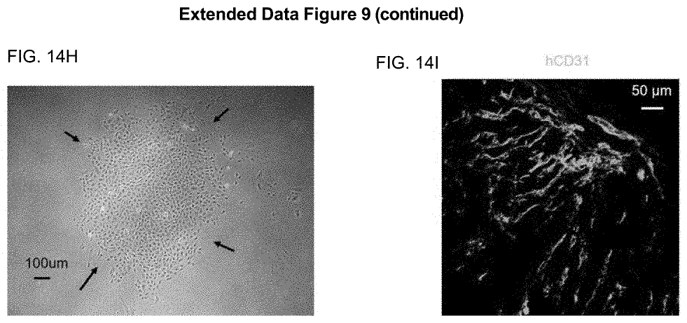

Abcg2-expressing VESC maintain blood vessels in adult mice. Since Abcg2-VESC derived TdTomato+ EC not only contribute to vessel growth but are also maintained in the adult vessels throughout life (FIG. 1), and Abcg2-VESC have self-renew potential (FIG. 2g), we looked to see if these cells were also maintained in blood vessels of adult animals. Indeed, when tamoxifen was administered into 6-8-week-old adult ABCG2TT mice (FIG. 3a), single TdTomato⁺ EC could also be identified in multiple tissues include heart, lung, retina, skeletal muscle and skin (FIG. 3b-c, e, FIG. 9, Extended Data FIG. 4) after 24 hours. Like their neonatal counterparts, adult tissue Abcg2-VESC were also enriched in ECFC potential in vitro compared to TdTomato⁻EC (FIG. 3d). In adult heart, lung and muscle, the distribution of Abcg2− VESC was evident in veins and capillaries but less frequent in arteries in contrast to neonatal pups (FIG. 9, Extended Data FIG. 4a, 4c-d).

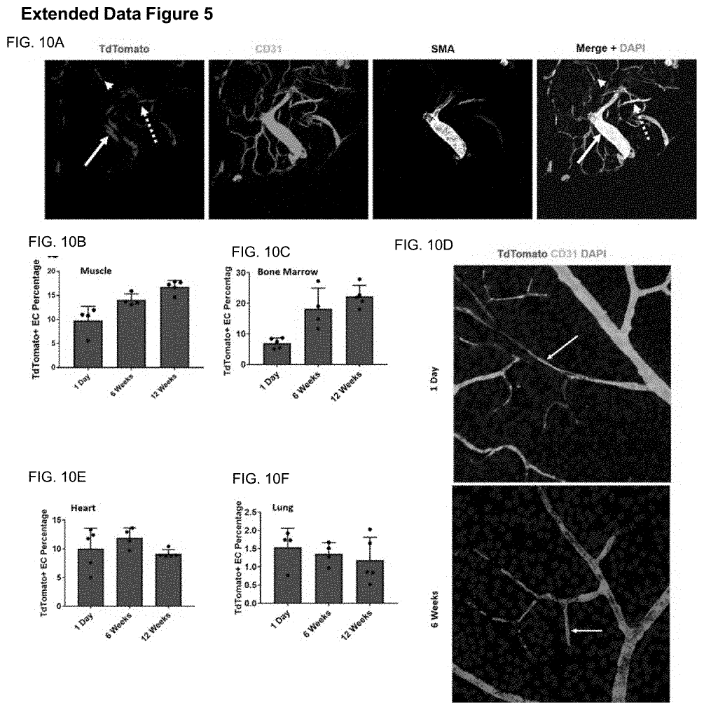

To study if these cells contribute to the maintenance of adult blood vessel endothelium, we gave a single tamoxifen injection to adult Abcg2TT mice and analyzed them after 6 or 12 weeks. Interestingly, in BM, skeletal muscle and skin, the percentage of Abcg2-VESC derived TdTomato+ EC steadily increased over the 12 weeks (FIG. 3e-h, FIG. 10A-10C, Extended Data FIG. 5a-c). Histologic analysis also confirmed that TdTomato+ EC single cells in skin 24 hours after tamoxifen injection formed TdTomato+ cell clusters or colonies overtime (FIG. 3e-g), proving that the increased TdTomato+ EC in adult vessels was derived from clonal expansion of Abcg2− VESC.

In contrast, Abcg2-EC in some adult tissues, like the retina, persisted as single cells for 3 months after tamoxifen injection (FIG. 10D, Extended Data FIG. 5d), which is in accordance with the previous finding that retinal EC display minimal turnover in adult life³³. Similarly, the percentage of TdTomato+ EC in adult mouse heart and lung did not significantly change in 3 months (FIG. 10E-10F, Extended Data FIG. 5e-f). However, Abcg2-VESC in adult heart did show superior in vitro colony forming potential (FIG. 3d). Additionally, previous studies using similar Abcg2CreERT mice have shown that EC labeled by Tamoxifen activation in adult mice led to robust contribution to vessel growth and regeneration after cardiac and skeletal muscle injury^{24,26}, though the source of the cells that gave rise to these labeled progeny were not identified^{24,26}. Thus, Abcg2-VESC in adult tissues like heart, though rarely proliferative in homeostatic conditions, may contribute to endothelial repair and replacement following injury. To directly test the potential of adult VESC to contribute to adult vessel regeneration upon injury, we induced experimental hind limb ischemia injury to nude mice and injected heart TdTomato⁺ EC of 6-week-old ABCG2TT mice collected 24 hours after a single tamoxifen injection. After 6 weeks, only 64.5±11.0% of blood flow in injured legs was restored in control mice, while the blood flow in injured legs of mice that received adult Abcg2-VESC injections was comparable with uninjured legs (103.00±18.58%, n=10 mice, FIG. 3i, FIG. 11A, Extended Data FIG. 6a). Importantly, robust adult VESC derived TdTomato⁺ blood vessels could be detected in the muscle tissue from all mice that received cell injections (FIG. 3j). These TdTomato⁺ vessels contributed to capillaries and major arteries that were larger than 100 μm in diameter (FIG. 11B, Extended Data FIG. 6b). Thus, Abcg2-VESC with the potential to regenerate capillaries, arterioles, and larger arteries in ischemic tissues are retained in adult tissue vasculature.

Previous reports have identified several putative VESC markers including CD157, ProCR, and cKit^{10,11,15}. However, most of these studies were focused on one or two organs and the relationship among those markers are not known. Thus, we analyzed heart, muscle and lung from Abcg2TT adult mice 24 hour after a single tamoxifen injection. Interestingly, in each organ, ProCR and cKit labeled the majority of EC, which included CD157 and TdTomato+ Abcg2-VESC, while Abcg2-VESC and CD157 VESC co-expression was rare (FIG. 12A-12F, Extended Data FIG. 7a-f). This data suggests that ProCR and cKit label a larger and more mature EC fraction, while CD157 and Abcg2 mark two somewhat distinct VESC fractions. Importantly, when tamoxifen was injected at P0 in Abcg2TT mice (FIG. 12G, Extended Data FIG. 7g), nearly 50% of adult CD157 EC were labeled by TdTomato (FIG. 12H-12J, Extended Data FIG. 7h-j), showing that neonatal Abcg2-VESC can give rise to different fractions of adult VESC, including CD157 expressing VESC.

### Example 5

Neonatal and adult Abcg2-expressing VESC have distinct gene expression signature. Next, we performed RNAseq analysis across multiple neonatal and adult organs to compare the gene expression between Abcg2-VESC and mature EC. In the neonatal heart, 3162 genes were differently expressed when comparing Abcg2− VESC and mature EC (1639 up, 1523 down; FIG. 13A, Extended Data FIG. 8a). Not surprisingly, the most differently expressed pathways were involved in proliferation and tissue development (FIG. 4a). Interestingly, pathways for extracellular matrix-receptor interaction and axon guidance, were also enriched in Abcg2-VESC (FIG. 4a), which is in accordance with the fact that these two pathways are crucial for angiogenesis^{34,35}. In addition, signaling pathways like Wnt, PI3K-Akt and cGMP-PKG, which are essential for the maintenance and function of stem cells, were also differentially enriched in Abcg2-VESC³⁶⁻³⁸ (FIG. 4a). Comparison of Abcg2-VESC and EC from two neonatal organs (lung and muscle) and two adult organs (heart and muscle) showed similar trends (FIG. 13B-13E, Extended Data FIG. 8b-e). In agreement with previous published data that highly angiogenic cells undergoing sprouting display some endothelial-to-mesenchymal transition (EnMT)^{11,14} genes, some EnMT, along with many angiogenesis genes were highly expressed in Abcg2-VESC (FIG. 13F-13G, Extended Data FIG. 8f-g). To find a gene expression signature of Abcg2-VESC across different neonatal and adult organs, we searched for the significant genes that were differently expressed in Abcg2-VESC in all 5 comparisons (FIG. 4b, FIG. 13H, Extended Data FIG. 8h) and validated using Q-RTPCR and flow cytometry (FIG. 13I, Extended Data FIG. 8i). In addition to Abcg2, most of the 34 commonly upregulated genes are involved in angiogenesis, proliferation regulation, and motility (FIG. 4b). Five members (Folsl2, Fos, Junb, Jund, Jun) of the activator protein 1 transcription factor family, which are known to regulate various cellular activities including proliferation, differentiation, EnMT, and apoptosis^{39,40}, were highly expressed in Abcg2-VESC in all neonatal and adult organs we tested (FIG. 4b). This result implies that this family of transcription factors plays important roles in the function of VESC.

While the gene expression analysis between putative VESC/VEPC and mature cells has been performed in several studies^{11,14,15,} most of these comparisons were completed in EC from a single adult organ and thus may have missed tissue/age specific differences. We collected transcriptome data from 3 neonatal and 2 adult organs, which enabled us to compare the difference in Abcg2-VESC gene expression among organs or between developmental stages. To our surprise, though Abcg2-VESC from organs at different developmental stages displayed some common gene expression patterns, the differences between Abcg2-VESC and mature EC in each group were dominated by specific gene expression signatures that the samples were more clustered based on organs and ages (FIG. 13I, Extended Data FIG. 8I). Compared to neonatal VESC, adult VESC showed decreased expression of cell cycle genes (FIG. 13M-13N, Extended Data FIG. 8m-n), suggesting adult VESC are more quiescent, which is in agreement with our lineage tracing data. We have previously published that EC from different organs express tissue specific signature transcription factors and angiocrine factors to perform various tissue specific functions and support different local cell types⁴¹. To test if VESC also possess tissue-specific features, we compared the expression of known lung, heart and muscle EC (FIG. 13O, Extended Data FIG. 8o) specific TF and AF (FIG. 4c, 4d, FIG. 13P-13Q, Extended Data FIG. 8p-q) in TdTomato+ VESC from neonatal organs. Indeed, VESC from each organ highly expressed their tissue-specific TF and AF (FIG. 4c, 4d, FIG. 13P-13Q, Extended Data FIG. 8p-q), suggesting that in addition to supporting vessel growth/maintenance, VESC in each organ also perform tissue-specific EC functions.

### Example 6

Human VESC are Labeled by ABCG2.

It has been known for a decade that human umbilical cord artery and vein contain EC with in vitro clonal colony forming potential⁵ However a marker that labels these colony forming cells prospectively is still lacking. We analyzed freshly isolated human umbilical cord arterial EC (HUAEC) and vein EC (HUVEC) for cell surface expression of previously published murine VESC/VEPC markers, including PROCR, CD157, CD34, and CKIT^{10,11,15,42}. Indeed, PROCR, CD157 and CD34 labeled nearly all human EC while ckit failed to label any EC (FIG. 15A-15C, Extended Data FIG. 10a-c), suggesting that none of these markers can be used to distinguish human VESC. To assess if ABCG2-VESC also exist in human vessels, we labeled freshly isolated human umbilical artery and vein samples with the 5D3 anti-ABCG2 monoclonal antibody^{17,43} and performed flow cytometry and immunofluorescent analysis. ABCG2⁺ EC are readily apparent in human blood vessel EC and represent 0.5 to 10%, respectively, of total human umbilical cord arterial EC (HUAEC) and vein EC (HUVEC) (FIG. 5b-5c). Similar to murine Abcg2+ VESC, which are maintained in adult tissues, ABCG2+EC could also be identified in adult human saphenous vein EC (FIG. 5d). When ABCG2⁺ cells from freshly isolated HUVEC were sorted by magnetic activated cell sorting (FIG. 14D, Extended Data FIG. 9d) and cultured in vitro, ABCG2⁺ HUVEC showed significantly superior ECFC potential than ABCG2⁻ HUVEC (FIG. 5e, FIG. 14E, Extended Data FIG. 9e). We isolated single ABCG2⁺ HUVEC derived cells, expanded them in vitro, and collected individual clones of ABCG2⁺ EC derived cells. We then transplanted the clones into NOD/SCID mice in the presence of OP9 stromal cells (4:1 ratio of EC to OP9) or OP9 cells that express NOTCH ligand DL-1 (OP9-DL1, 4:1 ratio of EC to OP9-DL1). We have previously reported that co-implantation of ECFC with OP9-DL1 cells promotes transplanted EC to adapt an arterial EC phenotype in vivo⁴⁴. After 2 weeks of implantation, robust perfused blood vessels were identified in all recovered gels (n=4, FIG. 5f, FIG. 14F, Extended Data FIG. 9f). While vessels from gels in which ABCG2+ HUVEC-derived EC were co-transplanted with OP9 displayed a capillary morphology (FIG. 14G, Extended Data FIG. 9g), single ABCG2⁺ HUVEC derived EC formed both capillaries and arteries in OP9-DL1 co-transplanted gels (FIG. 5g). As further confirmation, when single ABCG2⁺ HUVEC derived EC implanted gels were digested and re-plated, cobble-stone like secondary EC colonies were discovered from the culture (FIG. 14H, Extended Data FIG. 9h), and these cells could be re-implanted into secondary recipient mice to generate secondary donor vasculature (FIG. 14I, Extended Data FIG. 9i). In sum, like murine Abcg2− VESC, human ABCG2− VESC display the potential for clonal expansion in vitro, give rise to EC comprising capillaries and macrovessels in vivo, self-renew in vivo to form primary and secondary blood vessels, and thus fulfill the criteria of resident VESC.
1a Madisen, L. et al. A robust and high-throughput Cre reporting and characterization system for the whole mouse brain. Nature neuroscience 13, 133-140, doi:10.1038/nn.2467 (2010).
2a Fatima, S., Zhou, S. & Sorrentino, B. P. Abcg2 expression marks tissue-specific stem cells in multiple organs in a mouse progeny tracking model. Stem cells 30, 210-221, doi:10.1002/stem.1002 (2012).
3a Prasain, N. et al. Differentiation of human pluripotent stem cells to cells similar to cord-blood endothelial colony-forming cells. Nature biotechnology 32, 1151-1157, doi:10.1038/nbt.3048 (2014).
4a Yang, B. et al. Single-cell phenotyping within transparent intact tissue through whole-body clearing. Cell 158, 945-958, 25 doi:10.1016/j.cell.2014.07.017 (2014).
5a Winfree, S. et al. Quantitative Three-Dimensional Tissue Cytometry to Study Kidney Tissue and Resident Immune Cells. J Am Soc Nephrol 28, 2108-2118, doi:10.1681/ASN.2016091027 (2017).
6a Naito, H. et al. Endothelial Side Population Cells Contribute to Tumor Angiogenesis and Antiangiogenic Drug Resistance. Cancer research 76, 3200-3210, doi:10.1158/0008-5472.CAN-15-2998 (2016).
7a Zhou, S. et al. The ABC transporter Bcrp1/ABCG2 is expressed in a wide variety of stem cells and is a molecular determinant of the side-population phenotype. Nature medicine 7, 1028-1034, doi:10.1038/nm0901-1028 (2001).
8a Kobayashi, M. et al. Functional B-1 progenitor cells are present in the hematopoietic stem cell-deficient embryo and depend on Cbfbeta for their development. Proceedings of the National Academy of Sciences of the United States of America 111, 12151-12156, doi:10.1073/pnas.1407370111 (2014).
1 Lin, Y., Yoder, M. C. & Yoshimoto, M. Lymphoid progenitor emergence in the murine embryo and yolk sac precedes stem cell detection. Stem cells and development 23, 1168-1177, doi:10.1089/scd.2013.0536 (2014).
2 Barker, N. Adult intestinal stem cells: critical drivers of epithelial homeostasis and regeneration. Nature reviews. Molecular cell biology 15, 19-33, doi:10.1038/nrm3721 (2014).
3 Eaves, C. J. Hematopoietic stem cells: concepts, definitions, and the new reality. Blood 125, 332605-2613, doi:10.1182/blood-2014-12-570200 (2015).
4 Naito, H. et al. Endothelial Side Population Cells Contribute to Tumor Angiogenesis and Antiangiogenic Drug Resistance. Cancer research 76, 3200-3210, doi:10.1158/0008-5472.CAN-15-2998 (2016).
5 Ingram, D. A. et al. Vessel wall-derived endothelial cells rapidly proliferate because they contain a complete hierarchy of endothelial progenitor cells. Blood 105, 2783-2786, doi:10.1182/blood-2004-08-3057 (2005).
6 Alphonse, R. S. et al. The isolation and culture of endothelial colony-forming cells from human and rat lungs. Nature protocols 10, 1697-1708, doi:10.1038/nprot.2015.107 (2015).
7 Patel, J. et al. Functional Definition of Progenitors Versus Mature Endothelial Cells Reveals Key SoxF-Dependent Differentiation Process. Circulation 135, 786-805, 44doi: 10.1161/CIRCULATIONAHA. 116.024754 (2017).
8 Ingram, D. A. et al. Identification of a novel hierarchy of endothelial progenitor cells using human peripheral and umbilical cord blood. Blood 104, 2752-2760, doi:10.1182/blood-2004-04-1396 (2004).
9 Naito, H., Kidoya, H., Sakimoto, S., Wakabayashi, T. & Takakura, N. Identification and characterization of a resident vascular stem/progenitor cell population in preexisting blood vessels. The EM BO journal 31, 842-855, doi:10.1038/emboj.2011.465 (2012).
10 Fang, S., Wei, J., Pentinmikko, N., Leinonen, H. & Salven, P. Generation of functional blood vessels from a single c-kit+ adult vascular endothelial stem cell. PLoS biology 10, e1001407, doi: 10.1371/journal.pbio.1001407 (2012).
11 Yu, Q. C., Song, W., Wang, D. & Zeng, Y. A. Identification of blood vascular endothelial stem cells by the expression of protein C receptor. Cell research 26, 1079-1098, doi:10.1038/cr.2016.85 (2016).
12 Canete, A. et al. Characterization of a Fetal Liver Cell Population Endowed with Long-Term Multiorgan Endothelial Reconstitution Potential. Stem cells 35, 507-521, doi:10.1002/stem.2494 (2017).
13 Kusumbe, A. P., Ramasamy, S. K. & Adams, R. H. Coupling of angiogenesis and osteogenesis by a specific vessel subtype in bone. Nature 507, 323-328, doi:10.1038/nature13145 (2014).
14 Malinverno, M. et al. Peg3/PW1 Is a Marker of a Subset of Vessel Associated Endothelial Progenitors. Stem cells 35, 1328-1340, doi:10.1002/stem.2566 (2017).
15 Wakabayashi, T. et al. CD157 Marks Tissue-Resident Endothelial Stem Cells with Homeostatic and Regenerative Properties. Cell stem cell 22, 384-397 e386, doi:10.1016/j.stem.2018.01.010 (2018).
16 Challen, G. A. & Little, M. H. A side order of stem cells: the SP phenotype. Stem cells 24, 3-12, doi:10.1634/stemcells.2005-0116 (2006).
17 Zhou, S. et al. The ABC transporter Bcrp1/ABCG2 is expressed in a wide variety of stem cells and is a molecular determinant of the side-population phenotype. Nature medicine 7, 1028-1034, 2 doi:10.1038/nm0901-1028 (2001).
18 Zhou, S. et al. Bcrp1 gene expression is required for normal numbers of side population stem cells in mice, and confers relative protection to mitoxantrone in hematopoietic cells in vivo. Proceedings of the National Academy of Sciences of the United States of America 99, 12339-31 12344, doi:10.1073/pnas.192276999 (2002).
19 Zhou, S., Zong, Y., Lu, T. & Sorrentino, B. P. Hematopoietic cells from mice that are deficient in both Bcrp1/Abcg2 and Mdr1a/1b develop normally but are sensitized to mitoxantrone. BioTechniques 35, 1248-1252 (2003).
20 Bhattacharya, S., Das, A., Mallya, K. & Ahmad, I. Maintenance of retinal stem cells by Abcg2 is regulated by notch signaling. Journal of cell science 120, 2652-2662, doi:10.1242/jcs.008417 (2007).
21 Pfister, O. et al. Role of the ATP-binding cassette transporter Abcg2 in the phenotype and function of cardiac side population cells. Circulation research 103, 825-835, doi:10.1161/CIRCRESAHA.108.174615 (2008).
22 Chen, Z. et al. Suppression of ABCG2 inhibits cancer cell proliferation. International journal of cancer 126, 841-851, doi:10.1002/ijc.24796 (2010).
23 Higashikuni, Y. et al. The ATP-binding cassette transporter BCRP1/ABCG2 plays a pivotal role in cardiac repair after myocardial infarction via modulation of microvascular endothelial cell survival and function. Arteriosclerosis, thrombosis, and vascular biology 30, 2128-2135, doi:10.1161/ATVBAHA.110.211755 (2010).
24 Doyle, M. J. et al. Abcg2 labels multiple cell types in skeletal muscle and participates in muscle regeneration. The Journal of cell biology 195, 147-163, doi:10.1083/jcb.201103159 (2011).
25 Higashikuni, Y. et al. The ATP-binding cassette transporter ABCG2 protects against pressure overload-induced cardiac hypertrophy and heart failure by promoting angiogenesis and antioxidant response. Arteriosclerosis, thrombosis, and vascular biology 32, 654-661, doi:10.1161/ATVBAHA.111.240341 (2012).
26 Maher, T. J. et al. ATP-binding cassette transporter Abcg2 lineage contributes to the cardiac vasculature after oxidative stress. American journal of physiology. Heart and circulatory physiology 306, H1610-1618, doi:10.1152/ajpheart.00638.2013 (2014).
27 Fatima, S., Zhou, S. & Sorrentino, B. P. Abcg2 expression marks tissue-specific stem cells in multiple organs in a mouse progeny tracking model. Stem cells 30, 210-221, doi:10.1002/stem.1002 (2012).
28 Townsley, M. I. Structure and composition of pulmonary arteries, capillaries, and veins. Comprehensive Physiology 2, 675-709, doi:10.1002/cphy.c100081 (2012).
29 Sorensen, I., Adams, R. H. & Gossler, A. DLL1-mediated Notch activation regulates endothelial identity in mouse fetal arteries. Blood 113, 5680-5688, doi:10.1182/blood-2008-08-174508 (2009).
30 Uemura, A., Kusuhara, S., Katsuta, H. & Nishikawa, S. Angiogenesis in the mouse retina: a model system for experimental manipulation. Experimental cell research 312, 676-683, doi:10.1016/j.yexcr.2005.10.030 (2006).
31 Stalmans, I. et al. Arteriolar and venular patterning in retinas of mice selectively expressing VEGF isoforms. The Journal of clinical investigation 109, 327-336, doi:10.1172/JC114362 (2002).
32 Korn, C. & Augustin, H. G. Mechanisms of Vessel Pruning and Regression. Dev Cell 34, 5-17, doi:10.1016/j.devce1.2015.06.004 (2015).
33 Li Calzi, S., Neu, M. B., Shaw, L. C. & Grant, M. B. Endothelial progenitor dysfunction in the pathogenesis of diabetic retinopathy: treatment concept to correct diabetes-associated deficits. EPMA J 1, 88-100, doi:10.1007/s13167-010-0011-8 (2010).
34 Adams, R. H. & Eichmann, A. Axon guidance molecules in vascular patterning. Cold Spring Harb Perspect Biol 2, a001875, doi:10.1101/cshperspect.a001875 (2010).
35 Arroyo, A. G. & Iruela-Arispe, M. L. Extracellular matrix, inflammation, and the angiogenic response. Cardiovasc Res 86, 226-235, doi:10.1093/cvdcvq049 (2010).
36 Dejana, E. The role of wnt signaling in physiological and pathological angiogenesis. Circulation research 107, 943-952, doi:10.1161/CIRCRESAHA.110.223750 (2010).
37 Karar, J. & Maity, A. PI3K/AKT/mTOR Pathway in Angiogenesis. Front Mol Neurosci 4, 51, doi:10.3389/fnmol.2011.00051 (2011).
38 Aicher, A. et al. cGMP-dependent protein kinase I is crucial for angiogenesis and postnatal vasculogenesis. PloS one 4, e4879, doi:10.1371/journal.pone.0004879 (2009).
39 Jia, J. et al. AP-1 transcription factor mediates VEGF-induced endothelial cell migration and proliferation. Microvasc Res 105, 103-108, doi:10.1016/j.mvr.2016.02.004 (2016).
40 Shirali, A. S. et al. A multi-step transcriptional cascade underlies vascular regeneration in vivo. Scientific reports 8, 5430, doi:10.1038/s41598-018-23653-3 (2018).
41 Nolan, D. J. et al. Molecular signatures of tissue-specific microvascular endothelial cell heterogeneity in organ maintenance and regeneration. Dev Cell 26, 204-219, doi:10.1016/j.devce1.2013.06.017 (2013).
42 Yang, J. et al. CD34+ cells represent highly functional endothelial progenitor cells in murine bone marrow. PloS one 6, e20219, doi:10.1371/journal.pone.0020219 (2011).
43 Taylor, N. M. I. et al. Structure of the human multidrug transporter ABCG2. Nature 546, 504-509, doi:10.1038/nature22345 (2017).
44 Kim, H. et al. Notch ligand Delta-like 1 promotes in vivo vasculogenesis in human cord blood-derived endothelial colony forming cells. Cytotherapy 17, 579-592, doi:10.1016/j.jcyt.2014.12.003 (2015).
45 Alvarado-Moreno, J. A. et al. Endothelial colony-forming cells: Biological and functional abnormalities in patients with recurrent, unprovoked venous thromboembolic disease. Thrombosis research 137, 157-168, doi:10.1016/j.thromres.2015.11.005 (2016).
46 Prasain, N. et al. Differentiation of human pluripotent stem cells to cells similar to cord-blood endothelial colony-forming cells. Nature biotechnology 32, 1151-1157, doi:10.1038/nbt.3048 (2014).
47 Lee, J. Y. et al. Deregulation of Retinaldehyde Dehydrogenase 2 Leads to Defective Angiogenic Function of Endothelial Colony-Forming Cells in Pediatric Moyamoya Disease. Arteriosclerosis, thrombosis, and vascular biology 35, 1670-1677, doi:10.1161/ATVBAHA.115.305363 (2015).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

We claim:

1. A method for identifying and enriching a population of endothelial stem cells, comprising:
contacting a population of cells that includes endothelial stem cells with an agent that selectively binds to the cell surface marker ABCG2+; and
recovering at least a portion of endothelial stem cells that bind to the agent.

2. The method of claim 1, wherein the agent is an antibody.

3. The method of claim 2 wherein the antibody is linked to a bead.

4. The method of claim 3, wherein the bead is magnetic.

5. The method of claim 1, further comprising: isolating at least one endothelial stem cell that exhibits the ABCG2+ surface marker.

6. The method of claim 1, further comprising: creating a population of cells enriched in endothelial stem cells that exhibit the ABCG2+ surface marker.

7. The method of claim 5, further comprising: culturing the at least one endothelial stem cell that exhibits the ABCG2+ surface marker, in vitro.

8. The method of claim 1, wherein the endothelial stem cells are derived from human umbilical artery, umbilical vein, or saphenous vein.

9. The method of claim 1, wherein the endothelial stem cells are derived from murine umbilical artery, umbilical vein, or saphenous vein.

10-14. (canceled)

15. A medicament for the treatment of a patient, comprising: a population of ABCG2+ endothelial stem cells.

16. The medicament of claim 15, wherein the population of ABCG2+ endothelial stem cells was expanded ex vivo.

17. The medicament of claim 15, further comprising: at least one reagent that promotes the stabilization and/or promotes the growth of the ABCG2+ endothelial stem cells.

18. The medicament of claim 15, further comprising a collagen gel.

19. A method of treating a human or animal patient, comprising: administering at least one dose of a therapeutically effective amount of ABCG2+ endothelial stem cells to the patient.

20. The method of claim 19, wherein the ABCG2+ endothelial stem cells are suspended in a collagen gel, or in another matrix, or are in a container suitable for the delivery of the cells into the patient.

21. The method of claim 20, wherein the cells are suspended in a collagen gel and the therapeutically effective amount of ABCG2+ endothelial stem cells is on the order of more than two million cells per milliliter of collagen gel.

22. The method of claim 19, wherein the patient has been diagnosed with a condition that can benefit from development of an increase in vascular tissue.

23. The method of claim 19, wherein the patient exhibits at least one disease or defect selected from the group consisting of: peripheral arterial disease, critical limb ischemia, ischemic retinopathies, acute ischemic injury to kidney, and myocardial infarction.
